(19) United States
(12) Patent Application Publication
(10) Pub. No.: US 2023/0414657 A1
(43) Pub. Date: Dec. 28, 2023

(54) Novel compositions for neutralizing toxic effects of hydrogen peroxide in living cells or tissues

(71) Applicant: Dzala LLC, Tbilisi (GE)

(72) Inventors: Teymuras Kurzchalia, Berlin (DE); Veronica Diez, Dreseden (DE); Jan Prochazka, Lesetice (CZ); Vakhtang Pavlenishvili, Tbilisi (GE)

(73) Assignee: Dzala LLC, Tbilisi (GE)

(21) Appl. No.: 18/033,383

(22) PCT Filed: Jun. 7, 2021
(86) PCT No.: PCT/GE2021/050007

§ 371 Date: Apr. 24, 2023

(30) Foreign Application Priority Data
Nov. 10, 2020 (GE) AP 2020 15488

(51) Int. Cl. A61K 33/40; A61K 31/19; A61K 31/197; A61K 31/198; A61K 31/422; A61P 31/16; A61P 31/04; A61K 31/381; A61K 31/277; A23L 2/52; A23C 9/123; A23C 7/04

(52) U.S. Cl. CPC A61K 33/40; A61K 31/19; A61K 31/197; A61K 31/198; A61K 31/422; A61P 31/16; A61P 31/04; A61K 31/381; A61K 31/277; A23L 2/52; A23C 9/1234; A23C 7/043; A23V 2400/129; A23V 2400/137; A23V 2400/175; A23V 2400/231; A23V 2400/249

(57) ABSTRACT

The present invention relates to the field of food industry as well as to medicine, more particularly to novel compositions comprising hydrogen peroxide (H₂O₂) in combination with glycolic acid, glyoxylic acid, glycine, serine or salts thereof that allow using H₂O₂ as antimicrobial, especially antibacterial and antiviral or signaling agent yet neutralizing the toxic effects of the latter on the cell and tissues. The present invention also relates to a food product, an alcoholic/non-alcoholic beverage, or a nutritional supplement comprising the inventive composition and a method for production thereof.

### FIELD OF THE INVENTION

The present invention relates to the field of food industry as well as to medicine, more particularly to novel compositions comprising hydrogen peroxide (H₂O₂) in combination with glycolic acid, glyoxylic acid, glycine, serine or salts thereof that allow using H₂O₂ as antimicrobial, especially antibacterial and antiviral or signaling agent yet neutralizing the toxic effects of the latter on the cell and tissues. The present invention also relates to a food product, an alcoholic/non-alcoholic beverage, or a nutritional supplement comprising the inventive composition and a method for production thereof.

### BACKGROUND OF THE INVENTION

Notwithstanding the clearly shown progress in the way of achieving enhanced antioxidative actions of a variety of substances that can readily be used in food production to improve the health conditions of living beings, there still remains the need in a specific and effective method for defence against cellular toxicity of hydrogen peroxide that in the same time would allow usage of H₂O₂ as an antimicrobial or hormetic/signaling agent in all applications mentioned above.

Normal oxidative respiration of a cell in a living organism, especially mitochondrial respiration, entails generation of reactive oxygen species (ROS). The latter are generated also by redox enzymes, such as uncoupled nitric oxide synthase, cytochrome P-450 isoforms, and NADPH oxidase subtypes (NOXs), in the form of superoxide. When such short-lived ROS combine with nitric oxide (NO), the highly reactive peroxynitrite (a reactive nitrogen species [RNS]) are formed. Most importantly, superoxide can spontaneously or enzymatically be dismutated to form hydrogen peroxide and molecular oxygen. Hydrogen peroxide has a longer half-life than superoxide, and unlike superoxide, hydrogen peroxide can transfer across lipid membranes by either diffusion or transport through channels, such as aquaporins. For this reason, hydrogen peroxide, can cause oxidative damage to membrane lipids and even DNA and proteins if left unchecked by antioxidant systems.

When reacting with Fe2+(Fenton reaction—Fe⁺²+H₂O₂—Fe⁺³+HO⁻+HO), hydrogen peroxide leads to the formation of the highly reactive and damaging hydroxyl radical. Luckily, the Fenton reaction is limited within a cell, in part, by the lack of free transition metals, but may play a role in oxidative damage under an oxidative stress involving accumulation of high levels of intracellular hydrogen peroxide and liberation of Fe⁺² from intracellular storage sites.

While the above described is true for excess levels of hydrogen peroxide, low levels of the same, however, plays a role as a second messenger in signal transduction by modulating the oxidation state of redox-sensitive cysteines (Cys) to promote kinase function. Therefore, regulation of hydrogen peroxide is important to avoid formation of highly reactive and damaging radicals on the one hand, and to maintain its low level on the other hand.

Cells possess several antioxidant barriers, including enzymatic and non-enzymatic ones. It is known that enzymatic catabolism/inactivation of hydrogen peroxide could be executed by several classes of enzymes, like catalase, glutathione peroxidases (GPxs), peroxiredoxins and some others. Except catalase, almost all of these enzymes require the most abundant cellular ROS scavenger, glutathione, as cofactor. The latter exists in active, reduced (GSH, γ-glutamylcysteinylglycine) and inactive, oxidized states (GSSG). GSH is an obligate co-substrate in the reduction of hydrogen peroxide to water. For instance, GPx-1, one of the most abundant members of the GPx family of enzymes utilizes GSH as a cofactor to reduce hydrogen peroxide, resulting in the formation of oxidized glutathione (GSSG).

While glutathione protects against oxidative stress, the same substance, in excess, may also have deleterious effects due to a lack of essential cellular oxidants, which, it has been known for some time, can diminish cell growth responses and promote apoptotic pathways. Newer evidence points to additional cellular and physiological effects caused by lack of cellular oxidants, such as diminished mitochondrial function, and decreased cellular metabolism.

Generally speaking, antioxidants have the ability to donate electrons, which is the power being essential in neutralizing radicals and other reactive species. Accordingly, it seems quite logical that an antioxidant, once oxidized, loses its antioxidant power. This is true for glutathione notwithstanding its constant production. Even more problematic is that most people suffer from glutathione deficiency as a result of aging, stress, medication, and high toxicity levels in the blood. To enjoy the antioxidative benefits, it means that antioxidants should be permanently kept in reduced form or be supplemented with boosting pills, capsules, drips, injections, and even inhalants (e.g. glutathione). However, homeostasis of ROS is one of the mandatory requirements for maintaining the viability of the cellular system. Consequently, upon increasing antioxidant doses to permit the repair of pathological mitochondria, or under careless use of antioxidant doses, mitochondrial ROS may be disturbed and normal mitochondria may also be affected negatively as their levels of ROS may fall below their physiologically acceptable limit.

Besides GSH, several other antioxidants are applied. For instance, disclosed in US application US 2014/0335205 are materials and methods for the prevention and treatment of disease conditions associated with oxidative stress or a compromised reducing environment. The reducing agent, such as for example 5-aminosalicylic acid (5-ASA) and/or sodium thiosulfate and/or dihydro lipoic acid and/or alpha lipoic acid (ALA) and/or pyruvate can be an agent capable of reacting with H₂O₂. This agent can function intracellularly or it can be an extracellularly active agent or both. The extracellular activity, however, will also abolish antiseptic abilities of H₂O₂.

Another strategy in dealing with pathologies originating from mitochondrial dysfunction caused by mitochondrial ROS, is mitochondrial targeting of antioxidants. Some potent therapeutics have arisen from the group of mitochondria-targeted antioxidants, which specifically quench ROS in the organelle. However, despite very encouraging results in the use of mitochondria-targeted antioxidants, the mechanistic principle of delivering these agents is, to some extent, counterproductive. The main problem that arises is that injured mitochondria may carry a lower membrane potential when compared with normal ones and as a result, injured mitochondria are capable of taking up less therapeutic antioxidants than healthy mitochondria. Consequently, understanding the overall benefit of targeting dysfunctional mitochondria in pathological tissue requires furthering the development of alternative techniques to target mitochondria.

In an endeavor to regulate hydrogen peroxide and ensure its acceptable level within a cell, it is important to bear in mind that hydrogen peroxide still remains the effective antimicrobial agent and for this reason retaining its acceptable extracellular level is the matter of great importance. Hydrogen peroxide is used for sterilization of water and many raw materials for the food production (e.g. dairy products) and also as antibacterial agent for vegetable/fruit skin treatment (e.g. JPS2228224, JP2007053930). Although being a GRAS (Generally regarded as safe) substance, amounts used for the applications mentioned above are strictly regulated. For instance, pursuant to the Code of Federal Regulations (CHAPTER I—FOOD AND DRUG ADMINISTRATION DEPARTMENT OF HEALTH AND HUMAN SERVICES SUBCHAPTER B—FOOD FOR HUMAN CONSUMPTION), hydrogen peroxide as an antimicrobial agent is used to treat food only within the following specific limitations: milk, intended for use during the cheesemaking process as permitted in the appropriate standards of identity for cheese and related cheese products, whey, starch etc. Diluted solution of H₂O₂ is widely used in medical care as an antimicrobial agent for treatment of oral/nasal or outer ear cavities. It is also applied for disinfection of wounds.

Thus, there remains a need in the art for treatment and therapeutic modalities to provide biological instructions to one's cells that ensure reduction of glutathione, the main antioxidant found throughout the cytoplasm and mitochondria, alongside with its oxidation so as to avoid loss of its antioxidant power and, at the same time, to allow hydrogen peroxide to maintain its required concentration level in the extracellular space as an antimicrobial agent within a body.

It is object of the present invention to provide a composition comprising hydrogen peroxide (H₂O₂) in combination with glycolic acid, glyoxylic acid, glycine, serine or salts thereof that allow using H₂O₂ as antimicrobial, antiviral or signaling agent yet neutralizing the toxic effects of the latter on the cell and tissues. Especially, the composition is useful for prophylaxis or treatment of bacterial and viral diseases. Another object of the present invention is to provide a food product, an alcoholic/non-alcoholic beverage, or a nutritional supplement comprising the inventive composition.

The objective of the present invention is solved by the teaching of the independent claims. Further advantageous features, aspects and details of the invention are evident from the dependent claims, the description, the figures, and the examples of the present application.

### SUMMARY OF THE INVENTION

The present invention provides a novel composition that can be used to protect a cell, tissues, organs or a multi-cellular organism, such as animals and humans, against hydrogen peroxide induced oxidative stress but leaves antimicrobial and hormetic/signaling activities of H₂O₂ intact.

The present invention is based on ability of glycolic acid (GA) or its salt glycolate, to fully suppress the deleterious effects of peroxide on mitochondrial activity and growth of cells. This compound acts in the cell by entering serine-glycine metabolism where it is transformed sequentially into glyoxylic acid, glycine and serine. Inventors showed that the antioxidant capacity caused by glycolate depends on two interlocked metabolic pathways: serine-glycine and one-carbon metabolism. In this way, conversion of glycolate into glycine and serine ameliorates the drastically decreased NADPH/NADP⁺ and GSH/GSSG ratios induced by H₂O₂ treatment. Glyoxylic acid, glycine and serine have similar effects on mitochondrial activity and growth as glycolic acid. Remarkably, the above-mentioned effect of the glycolate is detected only in the presence of H₂O₂. It appears that H₂O₂ induces enzymes required for metabolism of glycolate and increased biosynthesis of NADPH. Thus, glycolate and its derivative on one hand and H₂O₂ on another, comprise a two-component system that neutralizes toxicity of H₂O₂ in the cell.

Thus, in one aspect, the present invention provides a novel composition that can be used to protect a cell, tissue, organ or an organism from H₂O₂ and comprises at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide.

In another aspect, the present invention provides for a drink solution or a capsule comprising at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide.

In another aspect, the present invention provides for a food product comprising at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide.

In still another aspect, the present invention provides for a non-alcoholic beverage comprising at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide.

In still another aspect, the present invention provides for an alcoholic beverage comprising at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide.

In still another aspect, the present invention provides for a nutritional supplement comprising at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide.

In still another aspect, the present invention provides for a method of preparing a food product comprising at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide, where component 2 is produced by an aerobic fermentation of milk.

In still another aspect, the present invention provides for a method of preparing a non-alcoholic beverage comprising at least two components, component 1 and component 2, wherein component 1 is selected from the group consisting of glycolic acid, glyoxylic acid, glycine, serine or salts thereof, and component 2 is selected from the group consisting of hydrogen peroxide or substances that are able to generate hydrogen peroxide.

In still another aspect, the present invention provides for a composition for neutralizing toxic effects of hydrogen peroxide in living cells or tissues, comprising at least two components, component 1 and component 2, wherein component 1 is selected from the group consisting of glycolic acid, glyoxylic acid, glycine, serine or salts thereof, and component 2 is hydrogen peroxide, for use as a medicament for prophylaxis or treatment of the nasal cavity and auricle bacterial and viral diseases.

In still another aspect, the present invention provides for a kit for neutralizing toxic effects of hydrogen peroxide in living cells or tissues, comprising at least two components, component 1 and component 2, wherein component 1 is selected from the group consisting of glycolic acid, glyoxylic acid, glycine, serine or salts thereof, and component 2 is hydrogen peroxide.

In still another aspect, the present invention provides for a kit comprising at least two components, component 1 and component 2, wherein component 1 is selected from the group consisting of glycolic acid, glyoxylic acid, glycine, serine or salts thereof, and component 2 is hydrogen peroxide, for use as a medicament for prophylaxis or treatment of the nasal cavity and auricle bacterial and viral diseases.

### DETAILED DESCRIPTION OF THE INVENTION

Oxidative stress causes profound alterations of various biological structures, including cellular membranes, lipids, proteins and nucleic acids, and it is involved in numerous malignancies. Reduced glutathione (GSH) is considered to be one of the most important scavengers of reactive oxygen species (ROS), and its ratio with oxidized glutathione (GSSG) may be used as a marker of oxidative stress. Within cells, total GSH exists free and bound to proteins. Since the enzyme glutathione reductase, which reverts free glutathione from its oxidized form (GSSG) is constitutively active and inducible upon oxidative stress, free glutathione exists almost exclusively in its reduced form. In a resting cell, the molar GSH:GSSG ratio exceeds 100:1, while in various models of oxidative stress, this ratio has been demonstrated to decrease to values of 10:1 and even 1:1.

The antioxidant function of GSH is primarily due to its involvement in enzymatic pathways that cells have developed against ROS. The most important pathway involves glutathione peroxidase (GPx) and glutathione reductase (GR). GPx catalyzes the reduction of hydrogen peroxide, which is produced by superoxide dismutase (SOD) through the dismutation of superoxide anions or organic hydroperoxides. GSH and GSH-dependent enzymes act in cooperation to scavenge ROS and/or neutralize their toxic oxidizing effect. These systems act at the same time and in cooperation to protect the human body from ROS. Under oxidative stress conditions, GSH is oxidized to GSSG; thus, the GSH:GSSG ratio is altered.

Oxidative stress is manifested by the excessive production of reactive oxygen species (ROS) in the face of insufficient or defective antioxidant defence systems. Oxidative stress causes profound alterations of various biological structures, including cellular membranes, lipids, proteins and nucleic acids.

Exposure of cells to hydrogen peroxide, one of the ROS, results in toxicity attributable to DNA damage by reactive oxygen species generated via the Fenton reaction:

H₂O₂+Fe⁺²+H⁺→Fe⁺³+[·OH]+H₂O.

The hydroxyl radical is an extremely powerful oxidant that reacts with most organic substrates at nearly diffusion-limited rates.

While it is true that reduced glutathione is responsible for attuning the antioxidant effects, it was shown that the redox state of nicotinamide adenine dinucleotidephosphate (NADPH) is another factor that modulates oxidative sensitivity. Cells resistant to oxidants are made in the conversion of NADP+ to NADPH. The latter is the major regulator of cellular redox potential and is crucial for the action of different antioxidant systems, including the maintenance of reduced glutathione pools. Thus, regeneration of NADPH from NADP⁺ is essential for protection against oxidative stress. Upon H₂O₂ challenge, the activity of the major sources of cellular NADPH, is induced by about 2.9-fold.

The pyridine nucleotide pools, nicotinamide adenine dinucleotide (NAD) and NADP, are crucial to the intracellular balance between the generation of reactive oxygen species (ROS) and their neutralization. The NAD pool participates in processes driving energy homoeostasis, generally associated with the subsequent production of ROS. The NADP pool, meanwhile, plays a primary role in maintaining the antioxidant defenses, but in some tissues may also serve as a cofactor in free radical generating reactions [1]. Both NAD and NADP act as “electron carriers”, ferrying reducing equivalents between redox reactions taking place inside the cell. In their oxidized forms, NAD⁺ and NADP⁺, both molecules may receive electrons by the addition of a hydride ion, producing the reduced forms, NADH and NADPH.

In its reduced form NADPH, represents a universal electron donor, not only to drive biosynthetic pathways. Perhaps even more importantly, NADPH is the unique provider of reducing equivalents to maintain or regenerate the cellular detoxifying and antioxidative defense systems. It is widely accepted that, in humans, the rate of NADPH regeneration is an indicative for protection against oxidative stress.

It has been observed by the inventors of the present invention that glycolic acid (GA) can restore the mitochondrial membrane potential in a living organism treated with an oxidant Paraquat (PQ²⁺) that produces superoxide (U.S. Pat. No. 10,434,077 B2). However, GA was unable to restore neither animal growth induced by PQ²⁺ exposure nor the OCR (Oxygen consumption Rate). The reason of above mentioned observation is that superoxide, can directly inactivate some Fe—S cluster-containing enzymes (e.g. aconitase) and it is also transformed into H₂O₂. In the present invention it is demonstrated that glycolate can combat massive oxidative stress induced by H₂O₂ and fully neutralize damage induced by it. It is also shown that GA is first converted into glycine and serine, thus entering serine/glycine metabolism. The serine-glycine metabolism is tightly connected with one-carbon metabolism. This way serine and glycine are becoming a source of 5,10-CH₂-THF (methylene-tetrahydrofolate), which is required for the regeneration of NADPH from NADP⁺. In this way, GA can ameliorate the drastically decreased NADPH/NADP⁺ and GSH/GSSG ratios induced by H₂O₂ treatment. Metabolization of GA in animals has been reported to occur mainly via its oxidation to glyoxylate, which can subsequently be transaminated to form glycine. In turn, glycine can undergo many transformations, among them, entering one-carbon metabolism (by donating a methyl group to tetrahydrofolate, THF) and the production of cysteine via conversion to serine. Serine/glycine metabolism has been proved to play a central role as a major provider of reducing equivalents to maintain cellular antioxidant systems and the fundamental function of glycolate as a natural antioxidant that improves cell fitness and survival. Effect of glycolic acid, on the other hand, depends on the activity of endogenous antioxidant systems and in particular on GSH. The studies showed also that GA supplementation increases the NADPH/NADP⁺ ratio, in this way stimulates the regeneration of reduced glutathione, resulting in restoration of the redox potential of the cell acting as a natural antioxidant. Thus, the results of the study clearly demonstrate that GA exerts its action by modulating a major antioxidant defense parameter, namely the ratio of GSH to GSSG (the reduced to the oxidized form of glutathione), one of the most common markers of oxidative stress and the extent of damage caused by it. Moreover, the inventors found that the accumulation of H₂O₂ was diminished in animals co-supplemented with peroxide and GA in comparison to those treated with peroxide alone.

Interestingly, it has been revealed that GA supplementation in the absence of H₂O₂ does not have a significant impact. In fact, only the combination of both compounds contributes to the induction of the GA-mediated antioxidant pathway.

It has been observed that H₂O₂ addition increases the oxidative stress of the animals while at the same time it is itself an up-regulator of this ROS-scavenging pathway which may work at its maximum efficiency when glycolate is externally supplied. In agreement with the observations of the inventors, it has been extensively reported the peroxide-mediated activation of the transcriptional regulator Nfr-2 and its ortholog that trigger the expression of antioxidant proteins and detoxification enzymes. Thus, H₂O₂ is as an inducer of the transcription factorNrf-2 and its ortholog Skn-1 that upregulate a plethora of phase II detoxification genes. One of the best known examples of genes induced by this transcription factor is gcs-1 encoding Glutamate Cysteine Ligase, the first enzyme in the biosynthesis of glutathione.

Thus, the inventors have identified a novel composition that is capable of protecting a cell, tissues, organs or a multi-cellular organism, such as animals and humans, against oxidative stress induced by H₂O₂. This composition is capable of generating an antioxidative defense by neutralizing ROS, particularly H₂O₂. Therefore, the present invention has several applications for disorders involving such an etiology.

According to one aspect, the present invention provides for a composition characterized by comprising at least two components, a component 1 and a component 2, wherein the component 1 is selected from the group consisting of glycolic acid, glyoxylic acid, glycine, serine or salts thereof, and the component 2 is selected from the group consisting of hydrogen peroxide or substances that are able to generate hydrogen peroxide.

In some embodiments, the composition for neutralizing toxic effects of hydrogen peroxide in living cells or tissues, comprises at least two components, a component 1 and a component 2, wherein the component 1 is selected from the group consisting of glycolic acid, glyoxylic acid, glycine, serine or salts thereof, and the component 2 is hydrogen peroxide.

In some embodiments, the composition for neutralizing toxic effects of hydrogen peroxide in living cells or tissues, comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 200 mM, preferably from 10 ppm to 150 mM, more preferably, from 10 ppm to 100 mM.

In case said composition is used as a antibacterial or antiviral agent for treatment of bacteria or viruses, said composition comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 0.1 mM to 150 mM, preferably, from 0.1 mM to 100 mM.

A molar ratio of component 2 and component 1 is in a range from 1:1 to 10000:1, preferred from 1:1 to 5000:1, more preferred from 1:1 to 2000:1, still more preferred from 1:1 to 1000:1, most preferred from 5:1 to 1000:1

In one embodiment, the present invention refers to a composition comprising at least two components, the component 1 and the component 2 as defined above, wherein the component 1 is glycolic acid, glyoxylic acid, or salt thereof and glycolic acid, glyoxylic acid, or salt thereof is presented in a concentration range between 5 mM and 20 mM, and the component 2 is presented in a concentration range from 0.1 mM to 150 mM.

Preferably, the composition as mention above, the glycolic acid, glyoxylic acid, or salt thereof is presented in a concentration range between 9 mM and 11 mM, more preferably, the concentration of glycolic acid or glyoxylic acid is 10 mM.

A molar ratio of hydrogen peroxide and glycolic acid, glyoxylic acid or salt thereof is in a range from 1:1 to 100:1, preferred from 1:1 to 50:1, more preferred from 1:1 to 20:1, still more preferred from 5:1 to 20:1, most preferred from 5:1 to 15:1

A salt of glycolic acid refers to a metal glycolate including not limited to sodium glycolate, potassium glycolate. Such glycolate salt may also form hydrate forms such as sodium glycolate monohydrate.

A salt of glyoxylic acid refers to a metal glyoxalate including not limited to sodium glyoxalate, potassium glyoxalate. Such glycolate salt may also form hydrate forms. such as sodium glyoxalate monohydrate

In one embodiment, the present invention refers to a composition comprising at least two components, the component 1 and the component 2, wherein the component 1 is glycine and glycine is presented in a concentration range between 0.1 mM and 2 mM, and the component 2 is hydrogen peroxide and presented in a concentration range from 0.1 mM to 150 mM.

A molar ratio of hydrogen peroxide and glycine, or salt thereof is in a range from 1:1 to 2000:1, preferred from 100:1 to 2000:1, more preferred from 500:1 to 1500:1, most preferred from 800:1 to 1200:1

In one embodiment, the present invention refers to a composition comprising at least two components, the component 1 and the component 2, wherein the component 1 is serine and serine is presented in a concentration range between 0.5 mM and 2.0 mM, and the component 2 is hydrogen peroxide and presented in a concentration range from 0.1 mM to 150 mM. Preferably serine is L-serine.

A molar ratio of hydrogen peroxide and serine, preferably L-serine, or salt thereof is in a range from 1:1 to 1000:1, preferred from 10:1 to 1000:1, more preferred from 100:1 to 500:1, most preferred from 150:1 to 250:1

In some embodiments, the present invention refers to a composition further comprising a component 3, the component 3 is selected from the group consisting of rupintrivir [also known as AG-7088, rupinavir or ethyl(E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate],2-(3,4-dichlorophenoxy)-5-nitrobenzonitrile (also known as MDL-860), benzothiophenes, and ((biphenyloxy)propyl) isoxazoles, preferably the component 3 is rupinitrivir.

Preferably, in the above-mentioned any of compositions, the component 3, i.e. the rupintrivir, or the 2-(3,4-dichloro-phenoxy)-5-nitrobenzonitrile or the benzothiophenes or the ((biphenyloxy)propyl) isoxazoles, is contained in the composition in a concentration range from 0.1 mM to 5 mM, more preferably 0.1 mM to 2 mM, still more preferably 0.1 mM to 1 mM, and most preferably 0.5 mM.

Preferably, in the above-mentioned any of compositions, a molar ratio of hydrogen peroxide and the component 3, i.e. rupintrivir or the 2-(3,4-dichloro-phenoxy)-5-nitrobenzonitrile or the benzothiophene derivative or the ((biphenyloxy)propyl) isoxazole derivative, or a salt thereof is in in a range from 1:1 to 1000:1, preferred from 10:1 to 1000:1, more preferred from 100:1 to 500:1, most preferred from 150:1 to 250:1

In an embodiment, the present invention refers to the composition for neutralizing toxic effects of hydrogen peroxide in living cells or tissues, comprises at least three components, a component 1, a component 2, and a component 3, wherein the component 1 is selected from the group consisting of glycolic acid, glyoxylic acid, glycine, serine or salts thereof; the component 2 is hydrogen peroxide; and the component 3 is rupintrivir or 2-(3,4-dichloro-phenoxy)-5-nitrobenzonitrile or the benzothiophene derivative or the ((biphenyloxy)propyl) isoxazole derivative.

In some embodiments, the composition for neutralizing toxic effects of hydrogen peroxide in living cells or tissues, comprises at least three components, a component 1, a component 2, and a component 3, wherein the component 1 is selected from the group consisting of glycolic acid, glyoxylic acid, glycine, serine or salts thereof in a concentration range from 0.1 mM to 20 mM; the component 2 is hydrogen peroxide in a concentration range from 10 ppm to 200 mM, preferably from 10 ppm to 150 mM, more preferably, from 10 ppm to 100 mM; and the component 3 is rupintrivir or 2-(3,4-dichloro-phenoxy)-5-nitrobenzonitrile or the benzothiophene derivative or the ((biphenyloxy)propyl) isoxazole derivative and presented in a concentration range from 0.1 mM to 5 mM, more preferably 0.1 mM to 2 mM, still more preferably 0.1 mM to 1 mM, and most preferably 0.5 mM.

The composition of the present invention as described herein as a drug can be prepared by any well-known method in a technical field of the pharmaceutics by mixing the component 1 and component 2 and a pharmacologically acceptable carrier appropriately.

The composition of the present invention as described herein further comprises one or more pharmacologically acceptable carriers. Examples of pharmacologically acceptable carriers include excipients (e.g., saccharides such as lactose, refined sugar, glucose, sucrose, mannitol, sorbitol and the like; starches of potato, wheat, corn, and the like; inorganic substances such as calcium carbonate, calcium sulfate, calcium phosphate, sodium hydrogen carbonate, sodium chloride, and the like; powders of plants such as licorice, gentian and the like), binders (e.g., polyvinyl alcohol, hydroxypropylcellulose, methylcellulose, ethylcellulose, carmellose, starchpaste liquid and the like), disintegrators (e.g., starch, agar, gelatine powder, microcrystallite cellulose, carmellosesodium, carmellose calcium, calcium carbonate, sodium hydrogencarbonate, sodium alginate and the like), lubricants (e.g. magnesium stearate, talc, hydrogenated vegetable oil, macrogol, silicon oil and the like), surfactants (e.g., fatty acidesters and the like), plasticizers (e.g., glycerin and the like), stabilizers (e.g., ˜-cyclodextrin and the like), fillers (e.g., silicon dioxides and the like), dispersants, suspenders, emulsifiers, diluents, buffers, antioxidants, bacteria inhibitors, preservatives, flavors, and the like.

The novel advantageous composition of the present invention may further contain, besides the component 1 and component 2, appropriate amounts of components (base, carrier, additive etc.) generally used for drug, quasi-drug, food and drink and the like, according to the use and dosage form of the antioxidant composition.

The components that can be appropriately added are not particularly limited and include, for example, vitamins, amino acids, alcohols, polyvalent alcohols, saccharides, polymer compounds such as gum substances and polysaccharides, surfactants, antiseptic, antibacterial, bactericidal agents, pH adjusters, chelating agents, antioxidants, enzyme components, binders, disintegrants, lubricants, fluidizers, algefacients, taste/odor correctives, coating agents, minerals, cellular stimulants, revitalizers, excipients, viscosity imparting agents, stabilizers, preservatives, isotonicity agents, dispersing agents, adsorbents, disintegration aids, wetting agents or moistening regulators, moisture-proof agents, colorants, flavoring agents or flavors, aromatics, reducing agents, solubilizing agents, bubbling agents, thickeners, solvents, bases, emulsifiers, plasticizers, buffering agents, gloss agents, fats and oils, moisturizing agents, UV absorbers and the like.

Examples of the vitamins include vitamin A, B, C, D, E, K, folic acid, pantothenic acid, nicotinamide, carnitine, choline, inositol, biotin, and the like.

Examples of the amino acids include leucine, isoleucine, valine, methionine, threonine, alanine, phenylalanine, tryptophan, lysine, glycine, asparagine, aspartic acid, serine, glutamine, proline, tyrosine, cysteine, histidine, omithine, hydroxyproline, hydroxylysine, glycylglycine, aminoethyl sulfonic acid, and pharmacologically acceptable salt thereof, and the like.

Examples of the alcohols include ethanol, isopropanol, lauryl alcohol, cetanol, stearyl alcohol, oleyl alcohol, lanolin alcohol, behenyl alcohol, 2-hexyldecanol, isostearyl alcohol, 2-octyldodecanol, and the like.

Examples of the polyvalent alcohols include ethyleneglycol, diethylene glycol, triethylene glycol, propylene glycol, polypropylene glycol, 1,3-butylene glycol, ethylene glycol monoethyl ether, ethylene glycol monobutyl ether, diethylene glycol monomethyl ether, diethylene glycolmonoethyl ether, polyethylene glycol, glycerin, pentaerythritol, sorbitol, mannitol, xylitol, inositol, and the like.

Examples of the saccharides include glucose, fructose, galactose, mannose, ribose, arabinose, xylose, deoxyribose, maltose, trehalose, sucrose, lactose, lactulose, raffinose, maltitol, erythritol, mannitol, xylitol, sorbitol, refined sugar, and the like.

These saccharides also include the derivative thereof, for example, phosphates (for example, glucose 6-phosphate etc.) and oxidants (for example, galacturonic acid, glucuronic acid, mannuronic acid, etc.), and food hygienically, pharmacologically or cosmetically acceptable salt thereof, and the like.

Examples of the surfactants include polyoxyethylene, polyalkyl siloxane, sorbitanmonooleate, sorbitantrioleate, sorbitanmonostearate, sorbitanmonoisostearate, sorbitanmonolaurate, sorbitanmonopalmitate, sorbitansesquioleate, to name a few, and pharmacologically acceptable salt thereof, and the like.

Examples of the antiseptic, antibacterial, bacteriocidal agents include methyl parahydroxybenzoate, ethyl parahydroxybenzoate, propyl parahydroxybenzoate, butylparahydroxybenzoate, acrinol, benzalkonium chloride, benzethonium chloride, cetylpyridinium chloride, to name a few, orthophthalaldehyde, and food hygienically or pharmacologicallyacceptable salt thereof, and the like.

Examples of the pH adjusters include hydrogen chloride, sulfuric acid, lactic acid, acetic acid, citric acid, tartaricacid, malic acid˜succinic acid, oxalic acid, gluconic acid, fumaric acid, propionic acid, acetic acid, aspartic acid, epsilon aminocaproic acid, glutamic acid, aminoethylsulfonicacid, phosphoric acid, polyphosphoric acid, boric acid, glucono lactone, ammonium acetate, sodium bicarbonate, sodiumcarbonate, potassium hydrate, sodium hydroxide, calciumhydroxide, magnesium hydroxide, monoethanolamine, triethanolamine, diisopropanolamine, triisopropanolamine, lysine, borax, and food hygienically or pharmacologically acceptable salt thereof, and the like.

Examples of the chelating agents include edetic acid, citrate, polyphosphoric acid, metaphosphoric acid, ascorbic acid, succinic acid, phytic acid, 1-hydroxyethane-1,1-diphosphonic acid, and food hygienically or pharmacologically acceptable salt thereof, and the like.

Examples of the antioxidants include ascorbic acid and its derivative, erythorbic acid and its derivative, tocopherol and its derivatives; catechins such as carotene, lycopene; propyl gallate, tannic acid, epigallocatechin; polyphenol such as anthocyanine, butylhydroxytoluene, butylhydroxyanisole, p-hydroxyanisole, and food hygienically or pharmacologically acceptable salt thereof, and the like.

Examples of the enzyme components include lipase, amylase, endopeptidase, catalase, lysozyme, Superoxide dismutase, glutathione peroxidase, elastase, collagenase, gelatinase, chymotrypsin, and the like.

Examples of the binders include starch, dextrin, gummi arabicumpulveratum, gelatin, hydroxypropyl starch, methylcellulose, carboxymethylcellulose, sodium hydroxypropylcellulose, crystalline cellulose, ethyl cellulose, polyvinyl alcohol, polyvinyl ether, polyvinyl pyrrolidone, macrogol, tragacanth, hydroxypropyl methylcellulose, calcium citrate, dextrin, pectin, and the like.

Examples of the disintegrants include starch, hydroxypropyl starch, carboxymethyl cellulose sodium, carboxymethyl cellulose calcium, carboxymethyl cellulose, low substituted hydroxypropyl cellulose, crystalline cellulose, and the like.

Examples of lubricants include talc, wax, hydrogenated vegetable oil, sucrose fatty acid ester, magnesium stearate, calcium stearate, aluminum stearate, polyethylene glycol, silica, and the like.

Examples of the fluidizers include light anhydrous silicic acid, dried aluminum hydroxide gel, syntheticaluminum silicate, magnesium silicate, and the like.

Examples of the algefacients include essential oil or essential oil constituent such as 1-menthol, d-menthol, d-menthol, d-camphor, dl-camphor, d-borneol, dl-borneol, geraniol, eucalyptus oil, bergamot oil, fennel oil, mentha oil, cinnamon oil, rose oil, peppermint oil, and the like.

Examples of the taste/odor correctives or the coating agents include cacao powder, cinnamon powder, green tea powder, lactose, refined sugar, glucose, mannitol, menthol, camphor, borneol, geraniol, eucalyptus oil, bergamot oil, fennel oil, mentha oil, cinnamon oil, rose oil, peppermint oil, mannitol, xylitol, fragrance essential oil, and the like.

Though the amounts of components in the composition of the present invention may be decided appropriately in purpose of use or its preparation, the amount of the component 1, such as glycolic acid or glyoxylic acid, is generally between 5 mM and 20 mM, preferably between 9 mM and 11 mM; more preferably 10 mM; and the component 2 is presented between 1 mM and 100 mM.

In some embodiments, the present invention refers to a composition as described herein for use in prophylaxis or treatment of the nasal cavity and auricle bacterial and viral diseases.

In some embodiments, the present invention refers to the composition as described herein for use, wherein the composition is in a form of an aerosol.

In some embodiments, the present invention refers to the composition as described herein for use, wherein the composition is in a form of nasal drops.

In some embodiments, the present invention refers to the composition as described herein for use, wherein the composition is administered to a subject in need thereof in a therapeutically effective amount in oral rout.

In some embodiments, the present invention refers to the composition as described herein for medical use, wherein the composition is administered to a subject in need thereof in a therapeutically effective amount in rectal rout.

When the composition of the present invention as drug is administered to a human, the dose varies depending upon the administration mode, the subject's age, body weight, etc..

the composition of the present invention can be used as a drug not only for humans but also for nonhuman animals. Nonhuman animals include nonhuman mammals, birds, reptiles, amphibians, fish and the like.

The use of the composition of the present invention as a drug is not particularly limited as long as it is based on an antioxidant activity and, for example, they can be used for the prophylaxis, improvement and/or treatment of various diseases primarily caused by oxidation of substances in the body, preferably prophylaxis, and/or treatment of nasal cavity, bacterial or viral diseases.

In another aspect, the present invention is directed to a kit for neutralizing toxic effects of hydrogen peroxide in living cells or tissues, comprising at least two components, a component 1 and a component 2, wherein the component 1 is selected from the group consisting of glycolic acid, glyoxylic acid, glycine, serine or salts thereof, and the component 2 is hydrogen peroxide.

Preferably, the present invention is directed to a kit for neutralizing toxic effects of hydrogen peroxide in living cells or tissues, comprising at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 200 mM, preferably from 10 ppm to 150 mM, more preferably, from 10 ppm to 100 mM.

In some embodiments, the present invention refers to the kit comprising at least two components, the component 1 and the component 2 as defined above, wherein the component 1 is glycolic acid, glyoxylic acid, or salt thereof and glycolic acid, glyoxylic acid, or salt thereof is presented in a concentration range between 5 mM and 20 mM, and the component 2 is presented in a concentration range from 0.1 mM to 150 mM. Preferably, the kit as mention above, the glycolic acid, glyoxylic acid, or salt thereof is presented in a concentration range between 9 mM and 11 mM, more preferably, the concentration of glycolic acid or glyoxylic acid is 10 mM.

A molar ratio of hydrogen peroxide and glycolic acid, glyoxylic acid or salt thereof is in a range from 1:1 to 100:1, preferred from 1:1 to 50:1, more preferred from 1:1 to 20:1, still more preferred from 5:1 to 20:1, most preferred from 5:1 to 15:1

A salt of glycolic acid refers to a metal glycolate including not limited to sodium glycolate, potassium glycolate. Such glycolate salt may also form hydrate forms such as sodium glycolate monohydrate.

A salt of glyoxylic acid refers to a metal glyoxalate including not limited to sodium glyoxalate, potassium glyoxalate. Such glycolate salt may also form hydrate forms, such as sodium glyoxalate monohydrate.

In some embodiments, the present invention refers to the kit comprising at least two components, the component 1 and the component 2, wherein the component 1 is glycine and glycine is presented in a concentration range between 0.1 mM and 2 mM, and the component 2 is hydrogen peroxide and presented in a concentration range from 0.1 mM to 150 mM.

A molar ratio of hydrogen peroxide and glycine, or salt thereof is in a range from 1:1 to 2000:1, preferred from 100:1 to 2000:1, more preferred from 500:1 to 1500:1, most preferred from 800:1 to 1200:1

In some embodiments, the present invention refers to the kit comprising at least two components, the component 1 and the component 2, wherein the component 1 is serine and serine is presented in a concentration range between 0.5 mM and 2.0 mM, and the component 2 is hydrogen peroxide and presented in a concentration range from 0.1 mM to 150 mM. Preferably serine is L-serine.

A molar ratio of hydrogen peroxide and serine, preferably L-serine, or salt thereof is in a range from 1:1 to 1000:1, preferred from 10:1 to 1000:1, more preferred from 100:1 to 500:1, most preferred from 150:1 to 250:1 In some embodiments, the present invention refers to the kit further comprising rupintrivir or 2-(3,4-dichloro-phenoxy)-5-nitrobenzonitrile or a benzothiophene derivative or a ((biphenyloxy)propyl) isoxazole derivatives a component 3. Preferably, is presented in a concentration range from 0.1 mM to 5 mM, more preferably 0.1 mM to 2 mM, still more preferably 0.1 mM to 1 mM, and most preferably 0.5 mM.

A molar ratio of hydrogen peroxide and rupintrivir or 2-(3,4-dichloro-phenoxy)-5-nitrobenzonitrile or the benzothiophene derivative or the ((biphenyloxy)propyl) isoxazole derivative, or a salt thereof is in in a range from 1:1 to 1000:1, preferred from 10:1 to 1000:1, more preferred from 100:1 to 500:1, most preferred from 150:1 to 250:1

In an embodiment, the present invention refers to the kit for neutralizing toxic effects of hydrogen peroxide in living cells or tissues, comprises at least three components, a component 1, a component 2, and a component 3, wherein the component 1 is selected from the group consisting of glycolic acid, glyoxylic acid, glycine, serine or salts thereof; the component 2 is hydrogen peroxide; and the component 3 is selected from the group consisting of rupintrivir,2-(3,4-dichloro-phenoxy)-5-nitrobenzonitrile, the benzothiophenes, and the ((biphenyloxy)propyl) isoxazoles, preferably, the component 3 is rupintrivir.

In some embodiments, the kit for neutralizing toxic effects of hydrogen peroxide in living cells or tissues, comprises at least three components, a component 1, a component 2, and a component 3, wherein the component 1 is selected from the group consisting of glycolic acid, glyoxylic acid, glycine, serine or salts thereof in a concentration range from 0.1 mM to 20 mM; the component 2 is hydrogen peroxide in a concentration range from 10 ppm to 200 mM, preferably from 10 ppm to 150 mM, more preferably, from 10 ppm to 100 mM; and the component 3 is rupintrivir or 2-(3,4-dichloro-phenoxy)-5-nitrobenzonitrile or the benzothiophenes or the ((biphenyloxy)propyl) isoxazoles and presented in a concentration range from 0.1 mM to 5 mM, more preferably 0.1 mM to 2 mM, still more preferably 0.1 mM to 1 mM, and most preferably 0.5 mM.

The kit as described herein is useful for prophylaxis or treatment of bacterial and viral diseases.

The kit as described herein is useful for application to a subject in need thereof through oral, nasal, auricle, topical or rectal rout.

The kit as described herein is preferably used, wherein the component 1 and component 2 are administered to a subject in need thereof in sequence, the time period between administering the component 1 and the component 2 being 30 minutes at most.

Food Product/Beverage/Nutritional Supplement/

In another aspect of the present invention, the composition of the present invention is applied to a nutritional supplement, a food product or a beverage.

The composition of the present invention can be provided in the form of a drink solution or a capsule.

The food product according to the present invention can be dairy product or any other food.

The beverage according to the present invention can be a non-alcoholic beverage or an alcoholic beverage.

The non-alcoholic beverage according to the present invention is selected from a group consisting of still water, fruit juices and vegetable juices, carbonated drinks, and non-alcoholic beer.

The food or beverage of the present invention includes juices; soft drinks; alocoholic beverage; teas; lactic acid bacteria beverages; milk product such as fermented milk, butter, cheese, yogurt, processed milk and defatted milk; animal meat products such as ham, sausage and hamburger; fishcake products such as plate-like fish cake, egg products such as rolled egg with soup or dash˜maki in Japanese and egg tofu; confectioneries such as cookie, jelly, chewing gum, candy, drop and snack; frozen dessert such as ice cream, sherbert and iced lolly; breads; noodles; pickles; smoked food products; dried fishes; fishes boiled in soy sauce or tsukudani in Japanese; salt curing products; soups; condiments; food materials; food additives; or any other forms. The form of the food and beverage is not particularly limited, and may be in any form, such as solid, powdery, liquid, gel, and slurry forms, so far as it is in a form that is easily ingested. The food or beverage includes the feed for nonhuman animals.

Further the food or beverage of the present invention may be in the forms of powdery foods; sheet-like foods; bottled foods; bottled beverages; retort foods; capsule foods; tablet-like foods; fluid foods; nutritious supplement drinks or the like.

Ingesting a high concentration of hydrogen peroxide can lead to confusion, strokes, heart attacks, and clots in the lungs. Hydrogen peroxide, even at small amounts can release hundreds of milliliters of oxygen into the human body. If hydrogen peroxide is ingested, oxygen is formed and can get into the blood vessels and can finally just due to gravity enter the brain, the heart and/or the lungs. Ingesting a low amount of hydrogen peroxide can still lead to mild gastrointestinal effects and to tissue damage.

In addition it was surprisingly found that while H₂O₂ addition increases the oxidative stress of animals and humans, the same hydrogen peroxide concurrently is an upregulator of the ROS-scavenging pathway which works at its maximum efficiency when glycolic acid, glyoxylic acid, glycine or serine or salts of glycolic acid, glyoxylic acid, glycine or serine is externally supplied. In agreement with the observations of the inventors, H₂O₂ is as an inducer of the transcription factorNrf-2 and its ortholog Skn-1 that upregulate a plethora of phase II detoxification genes. For instance, it was surprisingly shown that the treatment of mice with an inventive composition of GA and hydrogen peroxide induces about 10% increase of their weight as seen in Example 3 and FIG. 9.

Thus, the Inventors have surprisingly found and shown that in apart to prevent or drastically decrease the adverse effects of hydrogen peroxide, in order to benefit from the positive effect of activating or up-regulating the ROS-scavenging pathway, it is actually not sufficient to administer only glycolic acid, glyoxylic acid, glycine, serine or a salt thereof. For these purposes it is actually required to administer glycolic acid, glyoxylic acid, glycine or serine or a salt of glycolic acid, glyoxylic acid, glycine or serine together with hydrogen peroxide, because only this combination of glycolic acid and hydrogen peroxide or glyoxylic acid and hydrogen peroxide or glycine and hydrogen peroxide or serine and hydrogen peroxide or a salt of glycolic acid and hydrogen peroxide or or a salt of glyoxylic acid and hydrogen peroxide or or a salt of glycine and hydrogen peroxide or or a salt of serine and hydrogen peroxide seems to be able to sufficiently activate or to sufficiently up-regulate the ROS-scavenging pathway.

Consequently, the compositions of the present invention comprise hydrogen peroxide together with component 1 and component 1 is selected from the group consisting of glycolic acid, glyoxylic acid, glycine, serine or a salt of glycolic acid, glyoxylic acid, glycine or serine.

The compositions of the present invention can be provided in form of the pure composition comprising or consisting of glycolic acid, glyoxylic acid, glycine, serine or a salt thereof as component 1 and hydrogen peroxide as component 2. Furthermore, the compositions of the present invention can also be provided as nutritional supplement, a food product or a beverage or a pharmaceutical formulation as disclosed herein.

In some embodiments, the present invention is directed to the food product, the alcoholic beverage, the non-alcoholic beverage or the nutritional supplement comprising the inventive composition, wherein, the inventive composition comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 200 mM, preferably from 10 ppm to 150 mM, more preferably, from 10 ppm to 100 mM.

Preferably, the present invention is directed to the food product, the alcoholic beverage, the non-alcoholic beverage or the nutritional supplement comprising the inventive composition, wherein, the inventive composition comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 1000 ppm, preferably from 10 ppm to 500 ppm, more preferably, from 10 ppm to 100 ppm, most preferably from 30 ppm to 50 ppm.

In some embodiments, the present invention is directed to the food product comprising the inventive composition, wherein, the inventive composition comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 1000 ppm, preferably from 10 ppm to 500 ppm, more preferably, from 10 ppm to 100 ppm, most preferably from 30 ppm to 50 ppm.

In some embodiments, the present invention is directed to the alcoholic beverage comprising the inventive composition, wherein, the inventive composition comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 1000 ppm, preferably from 10 ppm to 500 ppm, more preferably, from 10 ppm to 100 ppm, most preferably from 10 ppm to 50 ppm.

In some embodiments, the present invention is directed to the non-alcoholic beverage comprising the inventive composition, wherein, the inventive composition comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 1000 ppm, preferably from 10 ppm to 500 ppm, more preferably, from 10 ppm to 100 ppm, most preferably from 30 ppm to 50 ppm.

In some embodiments, the present invention is directed to the nutritional supplement comprising the inventive composition, wherein, the inventive composition comprises at least two components, a component 1 and a component 2, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 1000 ppm, preferably from 10 ppm to 500 ppm, more preferably, from 10 ppm to 100 ppm, most preferably from 30 ppm to 50 ppm.

In another aspect, the present invention refers to a method of preparing the food product as described herein, comprising the steps of:
Providing pasteurized milk and mix of bacterial starters for making Joghurt, Matsoni, Dahi, Bathora that comprise Individual bacterial cultures comprising the following bacterial strains: Lactobacillus coryniformis, Lactobacillus delbrueckii, Lactobacillus rhamnosus, Lactococcuslactis, and Streptococcus thermophiles;
Mixing the predetermined amount of said starter or bacterial culture with the milk and fermenting the mixture under aerobic conditions by contacting the milk with air;
Measuring the hydrogen peroxide concentration in the surface layer after forming the texture of the fermentation product with the thickness of up to substantially 2 cm;
Removing the surface layer upon achieving the hydrogen peroxide concentration within the range of from 30 ppm to 50 ppm and refrigerating the surface layer so as to reduce the fermentation process therein;
further fermenting the mass remaining after the step of removing the surface layer by repeating the preceding steps until the last surface layer has been fermented;
Combining all layers containing the hydrogen peroxide;
Adding the component 1 before or after the step of fermentation.

In another aspect, the present invention refers to a method of preparing the nonalcoholic beverage as described herein, comprising the steps of:
Providing pasteurized juice and mix of bacterial starters for making Joghurt, Matsoni, Dahi, Bathora that comprise Individual bacterial cultures comprising the following bacterial strains: Lactobacillus coryniformis, Lactobacillus delbrueckii, Lactobacillus rhamnosus, Lactococcuslactis, and Streptococcus thermophiles;
Mixing the predetermined amount of said starter or bacterial culture with the beverage and fermenting the mixture under aerobic conditions by contacting the beverage with air;
Measuring the hydrogen peroxide concentration within the mass of the fermented mixture;
Adding the component 1 when the hydrogen peroxide concentration reaches the amount of from 30 ppm to 50 ppm.

### DESCRIPTION OF FIGURES

FIG. 1. Glycolate supplementation maintains mitochondrial function and improves animal physiology of peroxide treated C. elegans worms. A) Mitochondrial membrane potential (Δψm-MMP) was measured in the different strains after 3 days of treatment via MitoTracker Red CMXROS staining and recording the fluorescence intensities. B) Respiration rates in terms of oxygen consumption rate (OCR) determined after 4 days of treatment using a Seahorse Analyzer and normalized to number of worms. C) Size of worms after 4 days of treatment, where the lengths in μm of at least 20 animals were averaged per condition in each experiment. In D, E and F, life span analysis without FUdR, brood size and embryonic lethality, respectively, is shown for worms that had been exposed to H₂O₂ and 10 mM GA, as indicated, during larval development. G) H₂O₂ levels in worms exposed to different conditions for 3 days. Then H₂O₂ accumulation was measured with AmplexRed and fluorescence intensity was normalized to protein levels. 100 mM H₂O₂, 10 mMglycolate (GA) and 0.5 mM BSO were used as indicated. Error bars represent standard error of the mean (n=3).

FIG. 2. Glycolate addition prevents the drop in the antioxidant capacity of glutathione caused by peroxide. A) Total glutathione (GSH+GSSG), B) GSSG and C) Ratio of GSH to GSSG levels in animals exposed to 100 mM H₂O₂ and supplemented or not with 10 mMglycolate (GA) and 0.5 mM L-buthioninesulfoximine (BSO). Values were determined after 3 days of treatment and normalized to number of worms. Error bars represent standard error of the mean (n=3). BSO was used as a substance that opposes the effect of glycolic acid and/or glycine. In the presence of BSO, as evident from FIG. 2, the biosynthesis of glutathione was inhibited so that glycolateor glycine cannot neutralize the toxic effect of hydrogen peroxide. Thus, adding BSO to glycolate or glycine will abolish the positive effect of glycolate and glycine and will lead to an increase of the oxidative stress.

FIG. 3. Metabolic pathway of glycolate in C. elegans. In red, genes used in this work. In bold, enzymes catalyzing each reaction. Dashed arrows indicate more than one enzymatic step. This diagram was constructed according to “glyoxylate and dicarboxylate metabolism” (cel00630), “glycine, serine and threonine metabolism” (cel00260) and “one-carbon pool by folate” (cel00670) from the KEGG pathway database and the metabolic network of glycolate, glycine and serine in WormFlux. GOX-1, glycolate oxidase 1; LDH-1, lactate dehydrogenase 1; GHPR-1, glyoxylate reductase/hydroxypyruvate reductase 1; AGTX, alanine-glyoxylate transaminase; Ala, L-alanine; Pyr, pyruvate; GCS, glycine cleavage system; MEL-32, serine hydroxymethyl transferase; MTHFD, methylene tetrahydrofolate reductase; GR, Glutathione reductase; 5,10-CH₂THF, 5,10-methylen tetrahydrofolate; 10-forTHF, 10-formyl tetrahydrofolate; THF, tetrahydrofolate.

FIG. 4. Glycine and L-serine supplementation rescue mitochondrial function and developmental rate of glycolate-oxidation-deficient worms. Mitochondrial membrane potential (A), respiration rates (B) and size (C) of wild type (N2) or mutant strains (ghpr-1 and ghpr-1, gox-1, Idh-1) exposed to 100 mM H₂O₂ and supplemented, with 10 mMglycolate, 100 μM glycine or 500 μM L-serine, as indicated. Measurements were carried out as described in FIG. 2. Error bars represent standard error of the mean (n=3).

FIG. 5. Glycine and L-serine but not glycolate can ameliorate the antioxidant capacity of C. elegans mutants unable to metabolize glycolate to glyoxylate. A) Total glutathione (GSH+GSSG) levels, B) GSSG levels and C) ratio of GSH to GSSG in wild type (N2), single (ghpr-1) and triple (ghpr-1, Idh-1, gox-1) mutant animals exposed to 100 mM H₂O₂ and supplemented or not with 10 mMglycolate (GA), 100 μM glycine or 500 μM L-serine. Values were determined after 3 days of treatment and normalized to number of worms. Error bars represent standard error of the mean (n=3).

FIG. 6. SHMT and GCS activities are essential for the restoration of mitochondrial function and antioxidant capacity mediated by glycine, L-serine and glycolate upon peroxide treatment. Mitochondrial membrane potential (A), respiration rates (B), size (C), and GSH/GSSG ratios (D) of wild type animals (N2) fed with empty vector (EV) or RNAi bacteria against mel-32 or gcst-1 genes exposed to 100 mM H₂O₂ and supplemented or not with 10 mMglycolate (GA), 100 μM glycine or 500 μM L-serine. Measurements were carried out as described in FIGS. 2 and 3. Error bars represent standard error of the mean (n=3).

FIG. 7. Glycolate supplementation, via serine-glycine metabolism, increases the ratio of NADPH to NADP⁺ of peroxide-treated worms. Ratio of NADPH to NADP⁺ quantified by LC-MS and normalized to protein levels in samples of in N2 worms treated as follows. A) Animals were exposed to peroxide together with glycolate, glycine or L-serine, as indicated, for 3 days. B) Worms fed with empty vector (EV) or RNAi bacteria against SHMT (mel-32) genes were incubated with H₂O₂ alone or together with glycolate for 3 days. Error bars represent standard error of the mean (n=4). 100 mM H₂O₂, 10 mMglycolate, 100 μM glycine and 500 μM L-serine.

FIG. 8. Scheme of the proposed pathway for the glycolate-mediated antioxidant activity. Upon exposure to a strong oxidant (e.g. Paraquat), superoxide (O₂⁻) is produced that in turn can generate H₂O₂ (see text). Both ROS molecules disturb the mitochondrial activity (oxygen consumption rate and mitochondrial membrane potential, ΔΨ) and animal growth rate. Exogenously added glycolate (GA) is converted into glycine and serine. This triggers the NADPH/NADP⁺ ratio that together with the generation of the building blocks for GSH synthesis, glycine and L-cysteine (the latter produced form L-serine), increase the GSH/GSSG ratio. The boosted GSH levels counteract the deleterious effects of H₂O₂ on mitochondrial function and growth but not those of superoxide itself.

FIG. 9. Influence of a Composition comprising GA and hydrogen peroxide on growth of mice. 4 groups of experimental animals of strain C57Bl_6NCtr, consisting of 11 males in each were established. P—treated with 0.25% hydrogen peroxide in drinking water; G—treated with 0.01M GA in drinking water; PG—treated with a composition of 0.25% hydrogen peroxide and 0.01M GA in drinking water; W—plain drinking water. Experiment were started when animals were 6 weeks old and continued until reaching 16^{th} week of age. Mice were weighted once a week.

FIG. 10. Antimicrobial effect of a composition comprising 0.5% glycine and 0.3% hydrogen peroxide. Figure shows percentage of bacterial colonies that survive after processing of bacterial culture with the composition after 5, 10 and 15 minutes (black bars). As seen more than 99% of bacterial colonies are eliminated within 15 minutes. The composition comprising glycine is as active as the 0.3% solution of hydrogen peroxide alone (white bars).

### EXAMPLES

The present invention is explained in more detail in the following by referring to examples, which are not to be constructed as limitative. In the following description,

### Example 1

### Materials and Methods

### Chemicals

Sodium glycolate (G0111, TCI), sodium D-lactate (71716, Sigma), glycine (G7126, Sigma), L-serine (S4500, Sigma), L-glutathione reduced (G4252, Sigma), L-buthioninesulfoximine (B2525, Sigma), hydrogen peroxide (H1009, Sigma), paraquat (sc-257968, SantaCruz biotechnologies or 36541 Fluka® from Sigma-Aldrich), IPTG (15529019, ThermoFisher) were used. [1-¹⁴C]-glycolate, [1-¹⁴C]-glycine and L-[1-¹⁴C]-serine were purchased from HARTMANN ANALYTIC (Braunschweig, Germany).

Worm Strains and Culture Conditions

C. elegans wild-type (N2) strain was received from Caenorhabditis Genetics Center, USA. Mutant strains were generated using the following primers. Idh-1: AATCAACAATTTTCATGTCT and TAAAAATCGCGCGCATTTGA; C31C9.2 (ghpr-1): TCTCGTATAAACAGAAAATATGG and GGGGCGCTCATTCTGGAAATTGGand F41E6.5 (gox-1): GAAGTTGCGTATGTCCTTCTandATAATTGTTTCGAATCATGG. The injection protocol consisted of a master mix of 15 μl (5 μM of each of the primers, Cas9-Protein NLS [12.5 μM], tracrRNA-IDT [12.5 μM], dpy10 Oligo-IDT [733 nM], dpy-10-sgRNA-IDT [2.5 μM] and Protein Buffer-stock [2-3×]) that was injected into young adults of N2 strain. Progeny of the rescued strains were genotyped for homozygous deletion with PCR primers for three generations. All mutants were outcrossed at least twice with the wild type to eliminate background mutations. Double and triple mutants were obtained by crossing single or double mutants and selecting the homozygous progeny by PCR.

Worms were maintained at 20° C. on nematode growth medium (NGM) agar plates seeded with Escherichia coli NA221¹. Gravid adults on NGM agar plates were treated with alkaline hypochlorite solution (i.e., bleached) to purify eggs. On the day prior to setting up the experiment, eggs obtained after bleaching were allowed to hatch overnight in M9 buffer to obtain synchronized L1 population.

For RNAi experiments in liquid, E. coli HT115 containing L4440 empty vector or the indicated cDNA fragment in L4440 was inoculated in 100 ml of LB containing 100 μg/ml ampicillin and incubated overnight at 37° C. in a shaking incubator at 250 rpm. To induce the production of dsRNA, a final concentration 0.8 mM IPTG was added to the collected bacterial pellet diluted 100-fold and incubated overnight at 25° C. in a shaking incubator at 250 rpm. Bacteria were then pelleted by centrifugation at 4000 rpm for 5 mins and resuspended in 200 μl S-medium containing 100 μg/ml ampicillin and 0.8 mM IPTG.

Peroxide Treatment

A population of synchronized L1 larvae was added to 30-ml glass tubes containing 2.5 ml of S-complete medium (liquid culture)² and NA22 or induced RNAi bacteria at an OD₆₀₀ of 1.9. 100 mM H₂O₂, 8 mM GA, 100 mM L-glycine, 500 mM L-serine and 1 mM BSO were added as indicated. Worms were incubated at 15° C. and 150 rpm in a shaking incubator for 2-6 days, refreshing the medium every 2 days.

Mitochondrial Staining and Microscopy

Mitochondrial staining was performed as described previously^{3,4}. Briefly, L1 worms were grown on plate or in liquid culture for 2-3 days at 15° C. Subsequently, they were pelleted in a 15-ml tube, rinsed twice with H₂O and resuspended in 100 μl of M9. 5 μM MitoTracker Red CMXRos (Thermo Fisher Scientific, Germany) in DMSO was added to the worm suspension and incubated in this solution for 1.5 hr at room temperature in the dark. Next, the excess dye was washed off with M9 buffer and worms were incubated in 100 μl M9 for 30 min to eliminate the excess dye in the gut. Finally, worms were paralyzed with 0.5 mM Levamizol (Sigma-Aldrich), placed on slides covered with a thin layer of 2% agarose on top of which the coverslip (22×22 mm, Menzel-Glaser no. 1) was fixed using nail polish. We used a Zeiss LSM 700 inverted laser scanning confocal microscope and a Zeiss LCI Plan-Neofluar 63×/1.3 ImmCorr DIC M27 objective to image mitochondria (Zeiss, Germany). MitoTracker Red CMXRos was excited at 555 nm and the emission above 560 nm was acquired by the second PMT. Optical sections at 0.1×0.1×0.5 μm³ x-y-z resolution were collected in a 4D hyperstack. Final images were adjusted for intensity and merged in Fiji. No non-linear adjustments were made.

H₂O₂ Measurements.

Hydrogen peroxide was measured using the Amplex Red hydrogen peroxide/peroxidase assay kit (Molecular Probes, Eugene, OR), adapting the protocol to C. elegans. In the presence of hydrogen peroxide, Amplex Red is oxidized by horseradish peroxidase to form a red-fluorescent oxidation product whose fluorescence intensity can be measured.

To measure ROS production from C. elegans, 5000-7000 L1 worms were exposed to each condition for 2-3 days as indicated and then washed four times in 1 ml of the reaction buffer supplied with the kit. Volumes were adjusted to 700-1200 nematodes/50 μl and 50 μl was pipetted into a 96-well plate. A total of 50 μl of the Amplex Red reaction buffer was then added to the wells, and after 1 hr fluorescence was measured with a fluorescence microplate reader using excitation at 530±12.5 nm and fluorescence detection at 590±17.5 nm. Background fluorescence, determined for a no-H₂O₂ control reaction, was subtracted from each value. The significance of differences between conditions was determined by an unpaired fest. GraphPadPrism 8.0 was used for these calculations. Three biological replicates were carried out for each condition.

Growth Rate Experiments

Synchronized L1 worms were incubated at 15° C. on plates or in liquid culture and exposed to different conditions as indicated. Single worms were selected randomly from the plates or aliquots were taken from the liquid cultures and added to NGM plates for immediate visualization. 25 worms per treatment were measured at 24-48 hr intervals and all growth curves were done four times. Images were analyzed using FIJI (Image J) software, with length measurements derived by the sum-total of a number of segmented lines of known length, down the center of the worm as described previously⁵.

Lifespan, Progeny Number and Embryonic Lethality Determination

Lifespan experiments were performed at 20° C. without fluorouracil, as described elsewhere (Liu, 2019). Briefly, for the treatment during larval development worms were incubated in liquid culture under different conditions from L1 until L4, and then transferred onto control NGM plates until death. During the reproductive period (days 1-8), worms were transferred to fresh plates every other day to separate them from their progeny. Survival was scored every other day throughout the lifespan and a worm was considered as dead when they did not respond to three taps. Worms that were missing, displaying internal egg hatching, losing vulva integrity, and burrowing into NGM agar were censored. Progeny number and embryonic lethality was determined the day after transferring the adult animals to fresh plates. Statistical analyses of lifespan were calculated by Log-rank (Mantel-Cox) tests on the Kaplan-Meier curves in GraphPad Prism. For progeny number and embryonic lethality, the significance of differences between conditions was determined using an unpaired t-test in GraphPadPrism 8.0. All experiments were performed in triplicate.

Oxygen Consumption Assay

Oxygen consumption of worms was measured with a Seahorse XFe96 system (Seahorse Bioscience, North Billerica, MA), as previously described⁴. L3 larvae of the different strains were grown on plates or in liquid culture and supplemented for 2-4 days as described for each experiment. After removing bacteria and debris, approximately 100 worms were pipetted into each well of a 96-well Seahorse XF^{e} assay plate and OCR was measured until it stabilized. Then, 3 subsequent measurements were made at 6.5-min intervals. Finally, the exact number of worms in each well was counted and used for normalization. A small number of abnormal readings were also filtered out at this stage. On average, 7-8 wells (technical replicates) were used for each condition. Normalized OCR values were averaged for the last 3 measurements for each strain and condition. Four biological replicates were analyzed for each condition.

Quantification of Glutathione

Sample preparation was performed as described previously with some modifications⁶. From each condition ˜10,000 synchronized animals were harvested and washed 3 times with sterile water. Worms were resuspended in PBS buffer and 5-μl aliquots were taken to estimate the total number of worms, after which they were vortexed and frozen immediately with liquid nitrogen. To thoroughly lyse the worms, 5 cycles of freezing and thawing were performed in a water bath at 37° C., followed by a 1-min sonication. The samples were then centrifuged and the supernatant tested for protein estimation and GSH quantification.

Total GSH, GSSG, and GSH/GSSG ratios were quantified using the GSH/GSSG-Glo assay kit (Promega) following the instructions of the manufacturer. Briefly, each sample was tested in triplicate in 96-well culture plates with total glutathione or GSSG lysis reagent, for total glutathione or GSSG measurement, respectively. The GSH probe, luciferin-NT, was converted to luciferin, which is coupled to a firefly luciferase. The plates were then read in an EnVisionLuminometer plate reader (Perkin Elmer, United States). The GSSG value was subtracted from the total glutathione to calculate GSH levels and GSH/GSSG ratio. Four biological replicates were analyzed for each condition.

NADPH and NADP⁺ Quantification

To measure NADP⁺ and NADPH from C. elegans, 10000-15000 L1 worms were exposed to each condition in duplicates for 3 days and then washed four times with H₂O. After taking an aliquot for protein estimation, worms were resuspended in 100 μl of H₂O and 230 μl of Eluent A (95% acetonitrile, 0.1 mM ammonium acetate and 0.01% NH40H) was added together with 1 μl of 1 μM chloropropamide as internal standard. 25 □M solutions of each nucleotide were treated in parallel as standards. Homogenization for 15 min at 4° C. and 300 g was carried out in a TissueLyser II (Qiagen) after adding ⅓ volume of 0.5 mm zirconium beads. The resulting mixture was centrifuged at 13,000 g and the supernatant transferred to a new tube.

LC-MS/MS analysis of the samples was performed on a high-performance liquid chromatography (HPLC) system (1200 Agilent) coupled online to G2-S QTOF (Waters). For normal phase chromatography Bridge Amide 3.5 ml (2.1×100 mm) column from Waters was used. The mobile phase composed of eluent A and eluent B (40% acetonitrile, 0.1 mM ammonium acetate and 0.01% NH40H) was applied with the following gradient program: Eluent B, from 0% to 100% within 18 min; 100% from 18 min to 21 min; 0% from 21 min to 26 min. The flow rate was set at 0.3 ml/min. The spray voltage was set at 3.0 kV and the source temperature was set at 120° C. Nitrogen was used as both cone gas (50 L/h) and desolvation gas (800 L/h), and argon as the collision gas. MS^{E} mode was used with Bridge Amide 3.5 □l (2.1×100 mm) column in ESI negative ionization polarity for the detection of the nucleosides. Mass chromatograms and mass spectral data were acquired and processed by MassLynx software (Waters). Three and four biological replicates were analyzed for each condition of mel-32 RNAi and EV, respectively.

Quantitative Real-Time PCR.

The differential expression of F41E6.5 (gox-1), C31C9.2 (ghpr-1), gcst-1, mel-32 and gcs-1 genes was measured using qRT-PCR. ˜5000 L1 N2 worms were exposed to each condition for 3 days at 15° Cas described above and then washed three times with H₂O. Total RNA was isolated using the RNeasy Mini Kit (Qiagen) and equal amounts of all samples were reverse transcribed with SuperScript Ill (Invitrogen) using oligo(dT) 12-18 primers. cDNA quality was tested using standard PCR. qRT-PCR was performed using pre-designed TaqMan Gene Expression Assay (ThermoFischer Scientific) with at least three samples in triplicates. For the relative quantification of gene expression levels, the 2^{-ΔΔCT} method and results were normalized to the expression level of the cdc-42 gene. Expression levels were averaged over technical and biological replicates,

Radioactive Labelling.

To quantify the uptake of glycolate, glycine and L-serine approximately 10000 synchronized L1 were incubated in liquid cultures as described above and grown for 3 days in the presence of 10 Ci of [1-¹⁴C]-glycolate, [1-¹⁴C]-glycine or L-[1-¹⁴C]-serine. At this stage, worms were snap-frozen and stored at 80° C. Animals were lysed by freezing/thawing and extracted by using the Bligh and Dyer method⁷. After phase separation, organic and aqueous fractions were recovered from the lower and upper phases, respectively. After drying and concentration, radioactivity was quantified using a scintillation counter and normalized for total number of worms.

Statistical Information

The number of biological replicates is stated above for each method. Results shown as means; error bars represent the standard error of the mean. The unpaired Student's t-test was used to determine statistical significance of differences between means (P<0.05 [*], P<0.01 [**], P<0.005 [***]) unless otherwise stated.

### Example 2: Glycolate Neutralizes the Toxic Effects of H₂O₂ on an Organism

Glycolate Counteracts the Deleterious Effects of H₂O₂ on Mitochondrial Function, Animal Reproductive Capacity and Lifespan

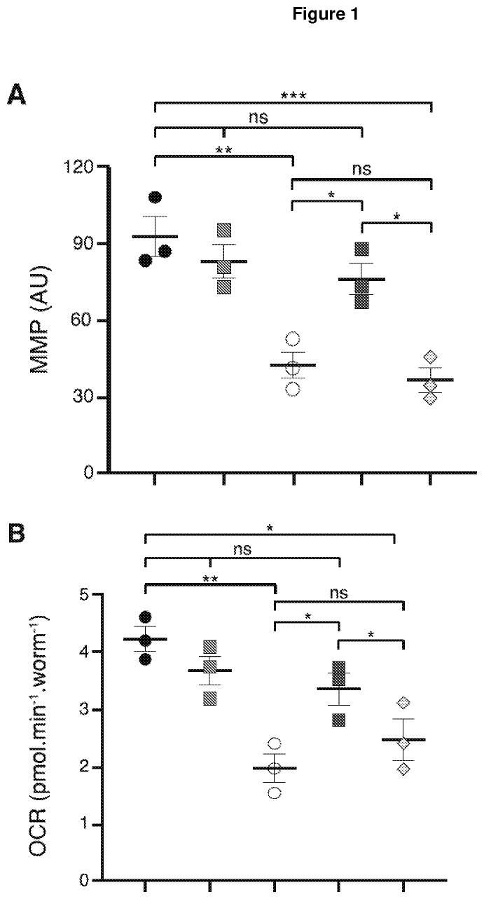

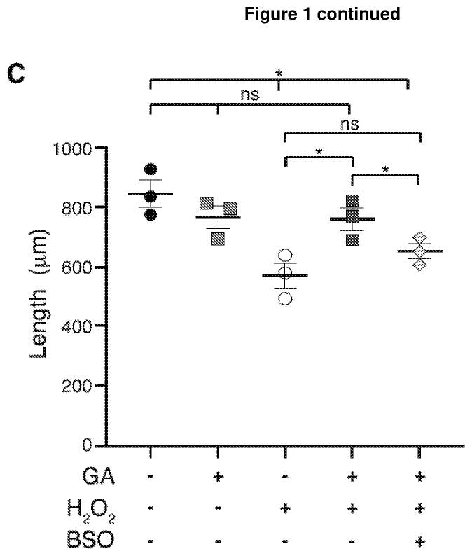

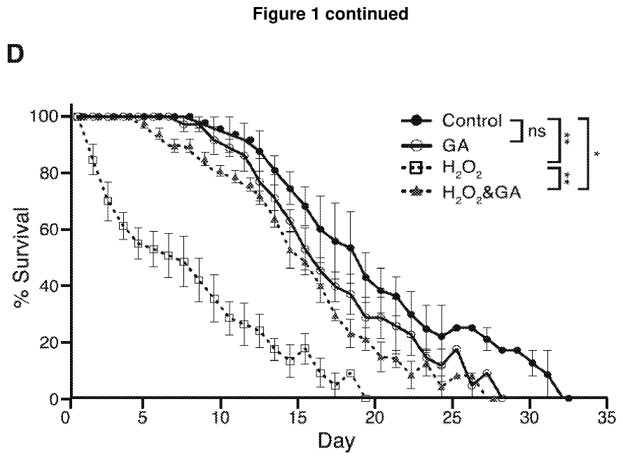

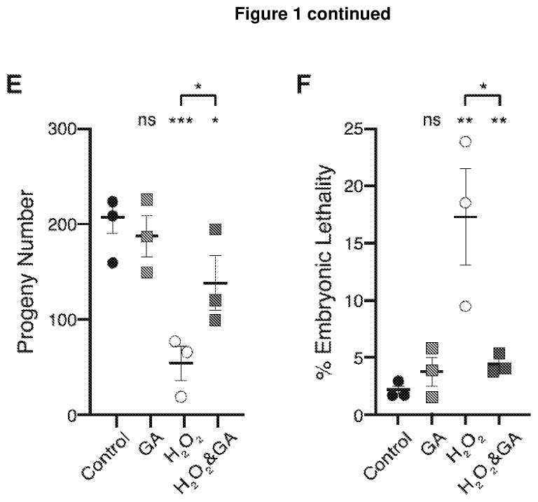

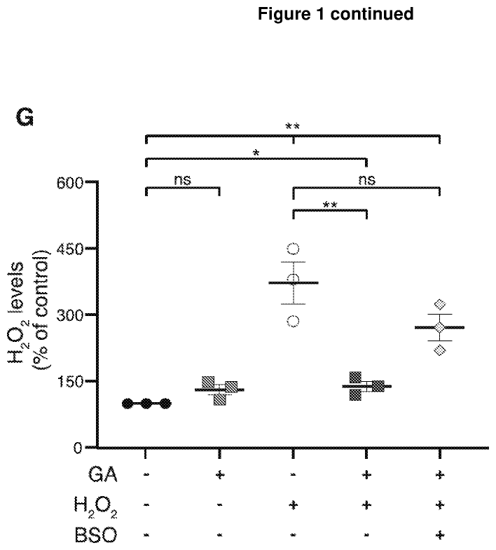

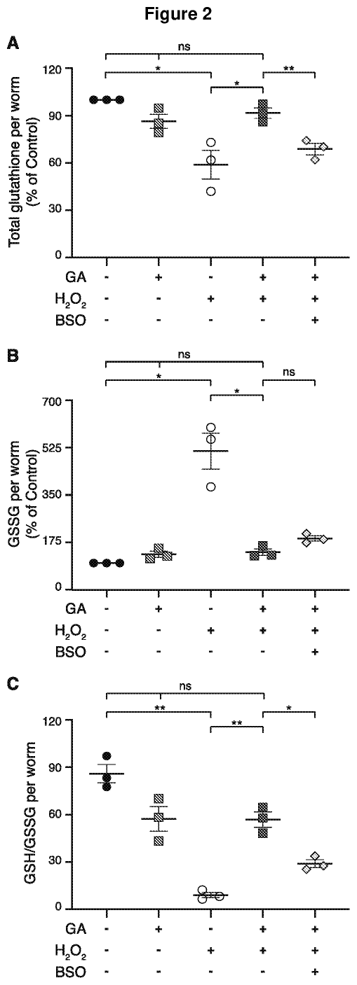

When larvae of the nematode C. elegans were treated with peroxide, there was a drop in MMP, but coincubation with GA prevented this effect (FIG. 1A). H₂O₂ also induced a decline in the respiration rate and a concomitant growth delay (FIGS. 1B and 1C). Remarkably, GA was able to restore both traits (FIGS. 2B and 2C). BSO treatment prevented the GA-mediated improvement in MMP, OCR and growth rate, thus strongly indicating that glutathione synthesis is fundamental for the observed oxidative stress relief mediated by GA (FIGS. 1A, 1B and 1C).

Notably, GAsupplementation also ameliorated the shortened lifespan and reduced reproductive capacity caused by H₂O₂. Animals exposed to H₂O₂ during larval stages and transferred to control plates as young adults showed a significantly reduced lifespan (Control worms: 18.5+/−1.19 days; H₂O₂ worms: 5.88+/−1.47 days [p=0.0006]). Addition of GA significantly increased lifespan (14.13+/−0.51 days [p=0.015]) making it comparable to that of untreated worms (FIG. 1D). A similar positive effect was observed when analyzing brood size and embryonic lethality in animals that had been treated only with H₂O₂ or in combination with GA (FIG. 1E and F, respectively).

Glycolate Supplementation Prevents the Drop in GSH/GSSG Ratio Caused by H₂O₂ Exposure

Glutathione is a tripeptide composed of glycine, cysteine and glutamate that coexists in two interconvertible forms: a reduced one, GSH, and an oxidized glutathione disulfide (GSSG), which is produced upon interaction with oxidative molecules. To test if GSH and GSSG concentrations are affected when treating C. elegans larvae with H₂O₂ and GA, glutathione levels upon exposure to peroxide were quantified (FIG. 2). It was observed that H₂O₂ treatment caused a significant decrease in total glutathione compared to non-treated animals, but this parameter was partially restored in the presence of GA (51% [p=0.0457] and 91% [p=0.1231] of the control, respectively). The effect of GA was again lost, however, if the inhibitor BSO was included in the medium (65% of the control [p=0.0051]) (FIG. 2A). More notable was the dramatic increase in the oxidized form of glutathione caused by H₂O₂, an effect that was also prevented by addition of GA (513% [p=0.0253] and 140% [p=0.0778] of the control, respectively) (FIG. 2B).

One of the most common markers of oxidative stress and the extent of damage caused by it is the ratio of the reduced to the oxidized form of glutathione (GSH/GSSG)⁸. Remarkably, it was obtained a 10-fold reduction in this parameter in worms incubated with peroxide alone but only a 28% decrease if GA was included in the medium (GSH/GSSH per worm=107.5 in control; 11.3 in H₂O₂ [p=0.0078] and 77.4 in H₂O₂+GA [p=0.103]). The effect of the latter was partially prevented by inhibition of glutathione synthesis with BSO (GSH/GSSG per worm=36 [p=0.012]) (FIG. 2C), in agreement with previously reported alterations of redox homeostasis caused by this inhibitor. These results clearly demonstrate that GA exerts its action by modulating a major antioxidant defense parameter, namely the ratio of GSH to GSSG. Moreover, the accumulation of H₂O₂ was diminished in animals co-supplemented with peroxide and GA in comparison to those treated with peroxide alone, but unchanged if glutathione synthesis was blocked by BSO (FIG. 1G).

Oxidation of Glycolate is a Prerequisite for its Activity

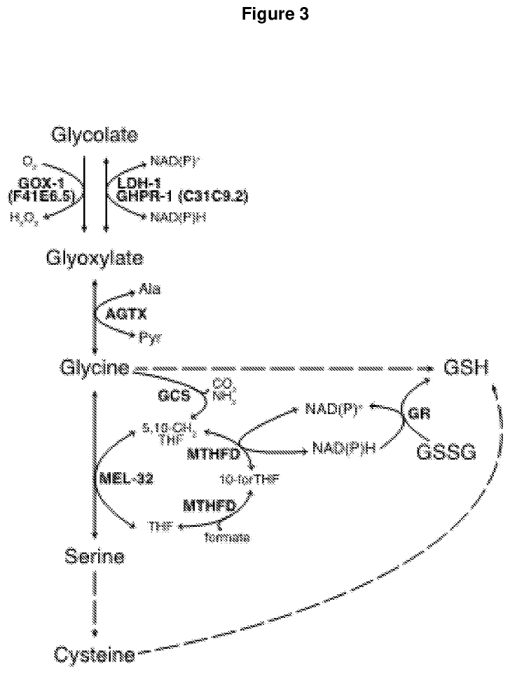

How treatment with GA can lead to an increase in GSH/GSSG ratio? Therefore, the underlying metabolic pathways that could link alterations in glutathione levels to GA itself or some of its downstream metabolites were explored. Metabolization of GA in animals has been reported to occur mainly via its oxidation to glyoxylate (FIG. 3)⁹⁻¹¹, which can subsequently be transaminated to form glycine. In turn, glycine can undergo many transformations, among them, entering one-carbon metabolism (by donating a methyl group to tetrahydrofolate, THF) and the production of cysteine via conversion to serine (FIG. 3, KEGG pathway database).

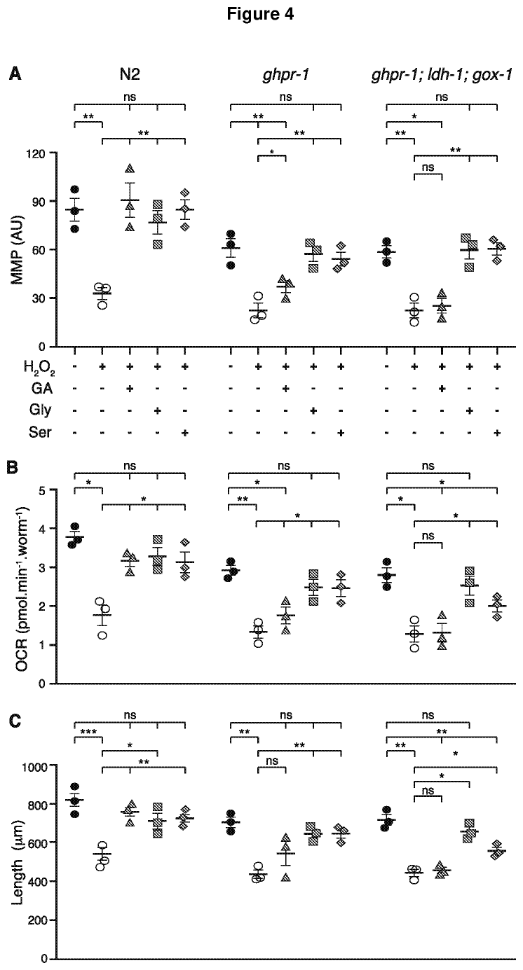

To test whether glycolate needs to be metabolized to exert its antioxidant activity, it was sought to obtain a mutant unable to catalyze the oxidation of glycolate to glyoxylate. In C. elegans, there are three enzymes that can be potentially responsible for this reaction (FIG. 3). The corresponding genes are: F41E6.5 (gox-1), encoding the ortholog of the human glycolate oxidase (HAO-1); Idh-1 (encoding the lactate dehydrogenase), which has been found to have glyoxylate reductase activity^{12,13}; and C31C9.2. The latter is predicted to encode the phosphoglycerate dehydrogenase involved in the biosynthesis of serine, but its sequence displays the highest homology to the human glyoxylate reductase/hydroxypyruvate reductase, reported to catalyze the interconversion of glyoxylate and glycolate¹⁴⁻¹⁷ (FIG. 3). From here, this gene will be designated as ghpr-1 (Glyoxylate/HydroxypyruvateReductase-1). Single mutants of gox-1 and Idl-1, or their double mutants showed no reduction in the effect of glycolate on MMP, OCR or growth. The deletion mutant of ghpr-1, however, displayed a significantly reduced effect of glycolate on MMP, oxygen consumption rate and developmental rate upon H₂O₂ treatment (FIG. 4A-4C). Remarkably, the glycolate-mediated protection was totally abolished in the ghpr-1; gox-1; Idh-1 triple mutant strain, demonstrating the overlapping activities of these three enzymes (FIG. 4A-4C).

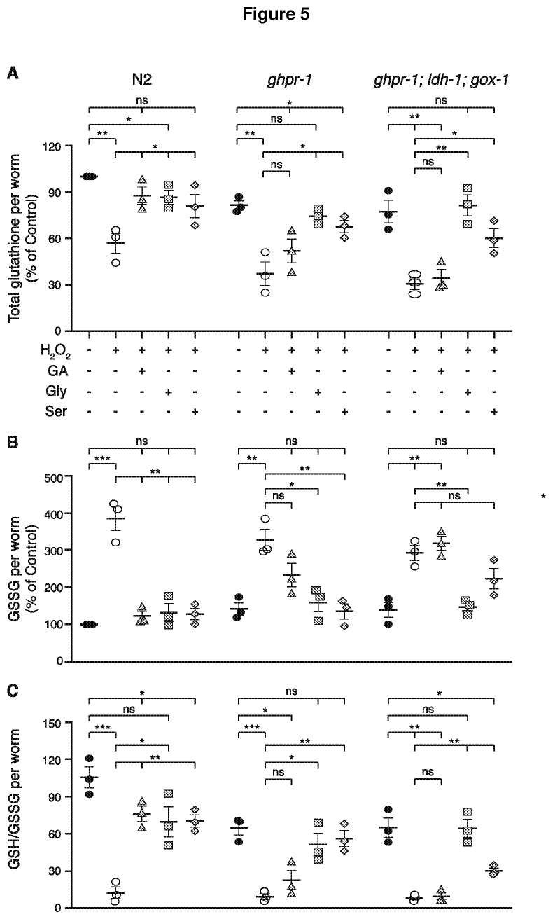

To test whether the reduction or absence of positive effects of GA on these mutants is due to deficient ROS-scavenging abilities, relative glutathione levels were quantified. Interestingly, both ghpr-1 and ghpr-1; gox-1; Idh-1 strains exhibited decreased levels of total glutathione compared to untreated N2 worms, a situation that was worsened by peroxide treatment but, in contrast to wild-type animals, unaffected by the addition of GA (FIG. 5A). Additionally, the significant increase in the oxidized form of the antioxidant, GSSG, observed in animals treated with peroxide, was not rescued by co-incubating the mutants with GA (FIG. 5B). A similar lack of response to GA was observed when analyzing the ratio of GSH to GSSG (FIG. 5C). These results clearly demonstrate that oxidation of GA is catalyzed mostly by GHPR-1 but also by GOX-1 and LDL-1 and suggest that further metabolization of this compound is crucial for its protective role on mitochondrial function and growth upon oxidative damage.

Relief of Oxidative Stress Requires Entry of Glycolate into Serine-Glycine Metabolism

According to the scheme in FIG. 3, glycolate can be converted into glycine and L-serine via the serine-glycine metabolic pathway. Can these amino acids also have a positive influence on C. elegans larvae exposed to peroxide? Indeed, both glycine and L-serine were able to restore MMP, respiration, growth and GSH/GSSG ratio to control levels when added to H₂O₂-treated worms (FIGS. 4 and 5). Moreover, both glycine and L-serine supplementation could overcome the deleterious effects of peroxide on strains deficient in the oxidation of glycolate (single ghpr-1 and ghpr-1; gox-1; Idh-1 triple mutants). Thus, these compounds are downstream effectors in the metabolic pathway of GA, as depicted in FIG. 3.

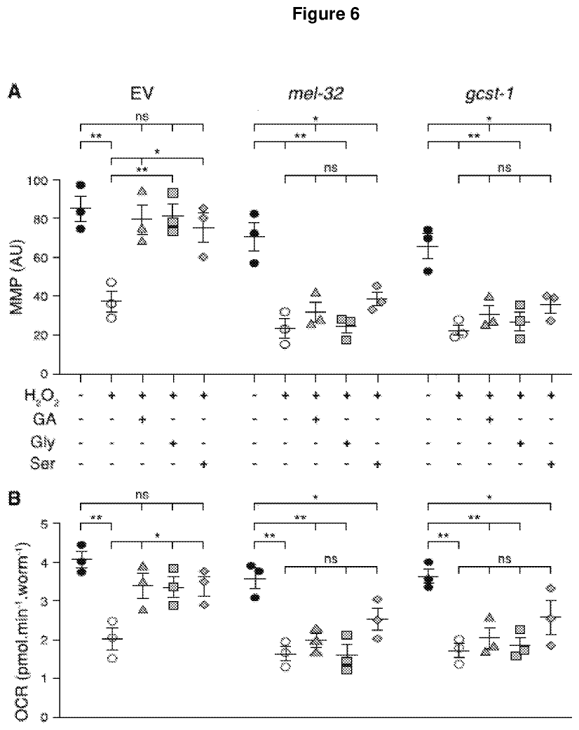

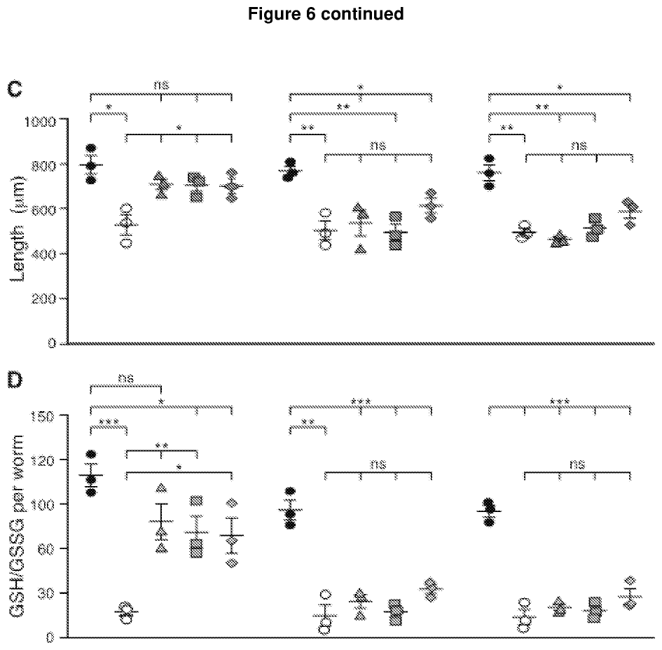

The next step was to understand the molecular mechanisms underlying the action of these two amino acids. Glycine is a component of the tripeptide glutathione, but it can also be converted into L-serine, which is a precursor of L-cysteine, another building block of GSH. However, more important perhaps is the conversion of glycine to L-serine that feeds one-carbon metabolism with THF as a key player (FIG. 3). Transformation of glycine into L-serine requires two enzymatic activities: one catalyzed by serine hydroxymethyltransferase (SHMT, encoded by mel-32) and the other by the glycine cleavage system (GCS, composed of the subunits T, P, L and H) (FIG. 3)¹⁸⁻²⁰. Through the action of GCS, glycine can provide a methyl group for the production of 5,10-methylene-H₄ folate (5,10-CH₂-THF), which together with another unit of glycine is a substrate of SHMT for the synthesis of L-serine (FIG. 3). The action of GA, glycine and serine on worms upon knock down of the SHMT and the GCS activities by RNAi against mel-32 or gcst-1 (encoding SHMT and subunit T of the GCS in C. elegans, respectively) was tested (FIG. 3). Remarkably, none of these three compounds were able to restore either MMP, OCR or growth rate, in contrast to the restored parameters exhibited in worms fed with empty vector RNAi bacteria (FIG. 6A-6C). Moreover, the GSH-to-GSSG ratio that was maintained close to control levels when worms fed with empty vector were grown in the presence of GA, glycine or serine (FIG. 6D), was strongly reduced in animals defective in either mel-32 or gcst-1 independently of their supplementation with any of these three metabolites (FIG. 6D). In addition, animals defective in glycolate oxidation (ghpr-1 and the ghpr-1, Idh1, gox-1 mutants) exhibited a loss of response to glycine and L-serine when fed with mel-32 RNAi bacteria. With this, it is demonstrated that functional SHMT and GCS activities are essential for the observed GA-, glycine- and serine-mediated protection against oxidative damage. Supporting this conclusion, life-span extension caused by GA supplementation of worms treated with peroxide was lost in mel-32-knockdown animals.

Note, GA supplementation in the absence of H₂O₂ does not have a significant impact on any of the parameters analyzed (FIGS. 1 and 2). This suggests that treatment with H₂O₂ could induce the expression of one or more of the proteins involved in the GA-metabolizing pathway (FIG. 3). In fact, it has long been established the role of H₂O₂ as inducer of the transcription factor Nrf-2 and its C. elegans ortholog Skn-1 that upregulate a plethora of phase II detoxification genes²¹⁻²⁵. One of the best known examples of genes induced by this transcription factor is gcs-1 encoding Glutamate Cysteine Ligase, the first enzyme in the biosynthesis of glutathione^{8,27,28}. Therefore, the relative expression levels of 5 different genes by RT-PCR upon peroxide or glycolate supplementation, including gcs-1 were analyzed. Remarkably, four of the tested genes (gox-1, ghpr-1, mel-32 and gcs-1) exhibited a significant induction when worms were exposed to different peroxide concentrations. The only exception was gcst-1, the expression of which was not significantly altered by the treatments. Unexpectedly, GA also moderately induced expression of these genes. This demonstrates that both compounds contribute to the regulation of the GA-mediated antioxidant pathway. Although, their specific roles could differ.

Glycolate Supplementation Restores the NADPH/NADP⁺ Ratio Impaired by Peroxide Treatment

Considering that both SHMT and GCS can produce 5,10-CH₂-THF, which is required for the regeneration of NADPH from NADP⁺ (FIG. 3), the most plausible explanation for the increase in GSH/GSSG ratio caused by GA is the alteration of NADPH levels. NADPH is the major regulator of cellular redox potential and is crucial for the action of different antioxidant systems, including the maintenance of reduced glutathione pools^{8,24,27} (FIG. 3). A series of recent publications has demonstrated that, in proliferating cells, the pentose phosphate pathway and the previously underestimated THF metabolism have nearly comparable contributions to the regeneration of NADPH from NADP⁺²⁹⁻³² (FIG. 3). Thus, GA and consequently glycine could increase NADPH by entering serine-glycine metabolism.

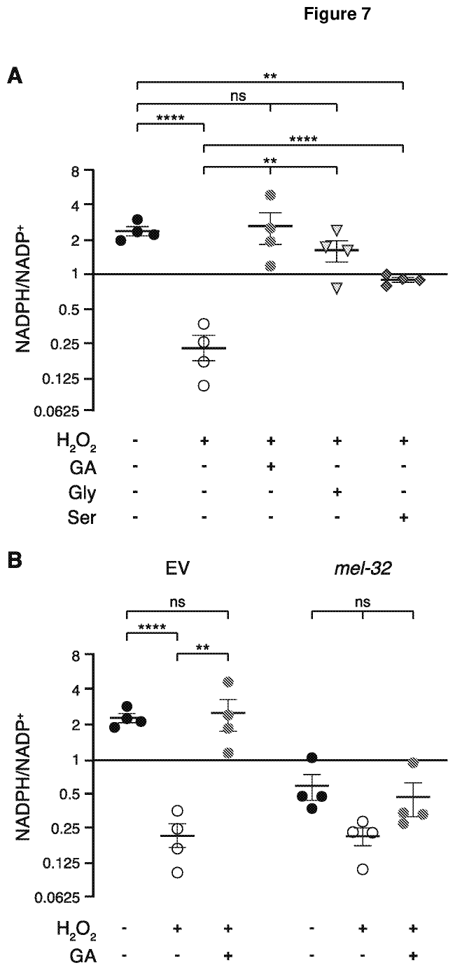

When subjected to H₂O₂ treatment, C. elegans larvae showed a significantly decreased ratio of NADPH to NADP⁺ (FIG. 7). However, when animals were supplemented with either GA or glycine in the presence of H₂O₂, the proportion of the reduced to the oxidized nucleotides was fully rescued (FIG. 7A), in agreement with the restoration of GSH/GSSG under the same conditions (FIG. 2C). L-serine supplementation, on the other hand, provoked a much weaker, but still significant, increase in the proportion of NADPH to NADP⁺ (FIG. 7A). This observation cannot be explained by a reduced uptake of L-serine by the worms compared to that of GA or glycine since radioactivity measured in animals labelled with [¹⁴C]L-serine was actually 3 times higher than in those labelled with [¹⁴C]GA. Instead, the modest effect of L-serine on NADPH levels could be due to the fact that in C. elegans, SHMT is prone to synthesize serine from glycine¹⁸. Nevertheless, excessive amounts of exogenously added L-serine most probably cause the shift of equilibrium towards glycine and thus generate the improved reducing capacity of the worms. In addition, the conversion of L-serine to L-cysteine (FIG. 3) could also have a positive impact on GSH levels without affecting the relative amounts of NADP⁺ and NADPH.

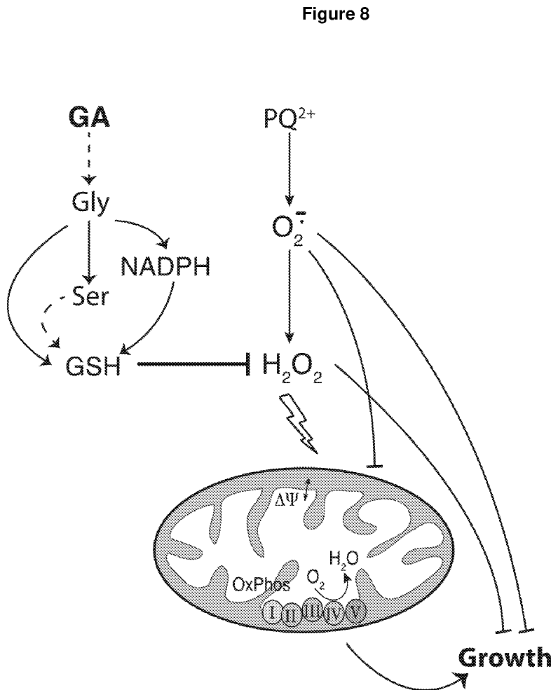

Consistent with the emerging contribution of one-carbon metabolism to the regeneration of NADPH, knocking down of the C. elegans SHMT enzyme, MEL-32, caused a reversal of the NADPH/NADP⁺ ratio compared with worms fed with empty vector (FIG. 7B). Exposure of these mel-32 knocked down worms to H₂O₂ produced a further decline in the proportion of the reduced to the oxidized nucleotides, which was not significantly affected by co-supplementation with GA. This correlates again with the ineffectiveness of GA addition on GSH/GSSG ratio in worms fed with mel-32 RNAi (FIGS. 6D and 7B). With this one can conclude that glycolate exerts its beneficial effects on C. elegans exposed to oxidative stress via the glycine-serine pathway that feeds one-carbon metabolism and the folate cycle, and in this way, improves the redox state of the animals (FIG. 8).

### Example 3: Influence of a Composition Comprising GA and Hydrogen Peroxide on Growth of Mice

The experimental treatment of wild-type mice (strain C57Bl_6NCtr) was performed as follows:

4 groups of experimental animals were established, 11 males in each group.
Group A: was treated with 0.25% hydrogen peroxide (H₂O₂) in drinking water
Group B: was treated with 0.01M GA in drinking water
Group C: was treated with a composition of 0.25% hydrogen peroxide (H₂O₂) and 0.01M GA in drinking water
Group D: was on plain drinking water

Experiment were started when animals were 6 weeks old and continued until reaching 16^{th} week of age. Mice were regularly weighted once a week.

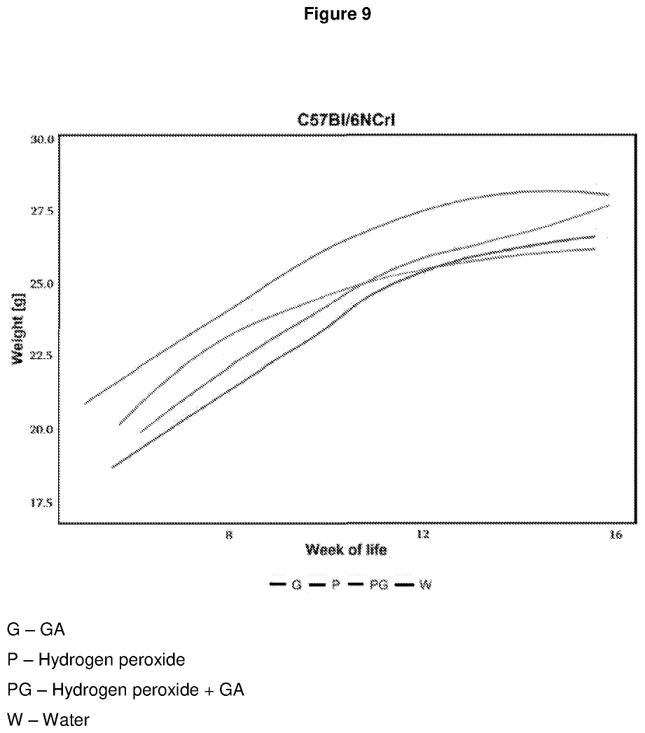

As can be seen in FIG. 9, the treatment of mice with a composition of GA and hydrogen peroxide induces about 10% increase of their weight. Remarkably, H₂O₂ alone has also a slight positive effect.

### Example 4: A Method of Preparing a Food Product Containing the Composition of the Present Invention

Pasteurized milk and mix of bacterial starters for making Joghurt, Matsoni, Dahi, Bathora that comprise Individual bacterial cultures comprising the following bacterial strains: Lactobacillus coryniformis, Lactobacillus delbrueckii, Lactobacillus rhamnosus, Lactococcuslactis, and Streptococcus thermophiles is provided. Next, the established amount known in the art of particular starter or bacterial culture is mixed with the milk and the mixture is fermented under aerobic conditions by contacting the milk with air. After forming the texture of the fermentation product with the thickness of up to substantially 2 cm, the hydrogen peroxide concentration in the surface layer is measured and upon achieving the hydrogen peroxide concentration within the range of 30 ppm to 50 ppm, the surface layer is removed and refrigerated so as to reduce the fermentation process therein. Then the mass remaining after the step of removing the surface layer is further fermented by repeating the preceding steps until the last surface layer has been fermented. Finally, all layers containing the hydrogen peroxide are combined. The component 1 is added before or after the step of fermentation.

### Example 5

A method of preparing a beverage containing the composition of the present invention

Pasteurized juice and mix of bacterial starters for making Joghurt, Matsoni, Dahi, Bathora that comprise Individual bacterial cultures comprising the following bacterial strains: Lactobacillus coryniformis, Lactobacillus delbrueckii, Lactobacillus rhamnosus, Lactococcuslactis, and Streptococcus thermophiles is provided. Next, the established amount known in the art of particular starter or bacterial culture is mixed with the juice and the mixture is fermented under aerobic conditions by contacting the juice with air;

the hydrogen peroxide concentration is measured within the mass of the fermented mixture, and the component 1 is added when the hydrogen peroxide concentration reaches the amount of 30 ppm to 50 ppm.

### Example 6: Anti-Viral Effect of Drug According to the Present Invention

The drug composition is tested according to the test of evaluation of the potential activity against a rhinovirus responsible for colds on fully differentiated human airway epithelial cells culture. The compositions are applied together with infectious virus and the incubation is carried out for 5 hours. 2 rinsing steps with the drug formulations are carried out. The remaining is collected and the RNA is dosed after cell lysis. The same collecting step is carried out 24 hours after and the same dosing step is carried out. The percentage expressed the changes in viral RNA, resulting in the 98% of inhibition of rhinovirus replication, leading to the anti-viral efficacy. In addition to the above described test, a kit comprising component 1 and component 2 was applied, wherein the component 2 was administered 15 minutes later than the component 1 was administered. The result in terms of inhibition of rhinovirus replication was the same as with the administration of the above mentioned drug composition.

### Example 7: Antimicrobial Effect of a Composition Comprising Hydrogen Peroxide and Glycine on Staphylococcus aureus Bacterial Culture, According to the Present Invention

A composition comprising 0.5% Glycine and 0.3% Hydrogen peroxide was prepared on 0.9% sodium chloride solution. Water was sterile purified. 4.5 ml of the composition was added to the sterile test tubes containing 0.5 ml of Staphylococcus aureus bacterial culture (ATCC 25923, compatible with McFarland standard 2) with titer of around 10⁶.

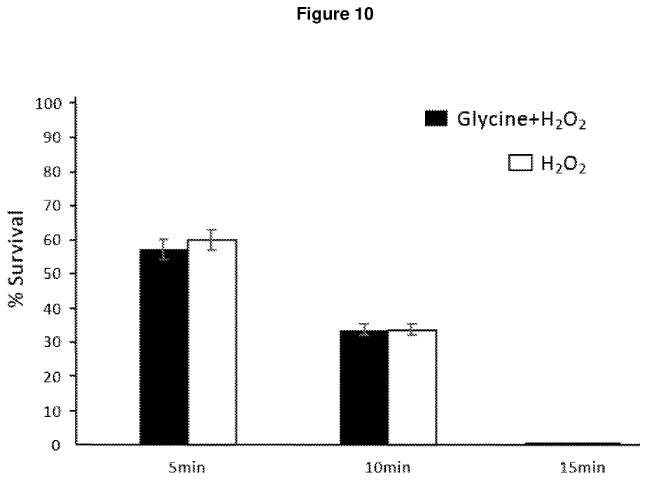

After desired times, hydrogen peroxide was neutralized by 1 ml of sterile 2.5% sodium bisulfite solution. (Previously it was demonstrated that 2.5% sodium bisulfite solution on itself had no antimicrobial effect). After incubation for 4-5 minutes, sample from each test tube was diluted 1000 times with sterile water, and 100 ml was spread evenly on standard tryptic soy agar petri plate. Plates were incubated at 37° C. for 24 hours and bacterial colonies were counted. As a positive control (100% survival), was taken a sample, where the composition was neutralized by sodium bisulfite instantly (FIG. 10).

### Example 8: Assessing Effects of Compositions Comprising GA, Glycine or Serine and Hydrogen Peroxide on Viability of Cells, According to the Present Invention

### Materials

Cells: T3M4 (Fast growing pancreatic cancer cells); Media: Standard medium —DMEM/10% FBS; Treatment medium—PBS/10% FBS.

### Experimental Setup

Cells were grown under standard condition and were seeded in 96-well plate (5000 cells per well). Treatment was started after 12 hrs. After replacement of Standard medium with Treatment medium, cells were treated with different doses of H₂O₂. For cell viability assessment 10 μl MTT solution (3-(4,5-dimethylthiazol-2-yl)-2,5-diphenyl tetrazolium bromide) were added into each well. In 3-4 hours medium was be aspirated. After the plate was dried, formed formazan was solubilized with isopropanol and reads of absorbance at 570 nm and 630 nm wavelengths were taken. Measurements at Time Zero (12 hrs.), Day-1 (+24 hrs.), Day-2 (+48 hrs) and Day-3 (+72 hrs.) were recorded. All values have been normalized and cell growth curve was build. Each treatment group was measured in quadruples and the experiment was repeated for 3 times.

As first, we identified toxic dose of H₂O₂. After this, we have continued with 2 selected H₂O₂ doses and applied different concentrations of GA, glycine or serine. We also had an additional control group, containing only the latter compounds. Groups at each time points have been statistically compared.

### Cyotoxicity Assessment Plate Example

| | Day 0 | | | | Day 1 | | | | Day 2 | | | |
|---|---|---|---|---|---|---|---|---|---|---|---|---|
| | 1 | 2 | 3 | 4 | 5 | 6 | 7 | 8 | 9 | 10 | 11 | 12 |
| A | Control/blank | | | | Control/blank | | | | Control/blank | | | |
| B | H₂O₂-dose 1 | | | | H₂O₂-dose 1 | | | | H₂O₂-dose 1 | | | |
| C | H₂O₂-dose 2 | | | | H₂O₂-dose 2 | | | | H₂O₂-dose 2 | | | |
| D | H₂O₂-dose 3 | | | | H₂O₂-dose 3 | | | | H₂O₂-dose 3 | | | |
| E | H₂O₂-dose 3 | | | | H₂O₂-dose 3 | | | | H₂O₂-dose 3 | | | |
| F | H₂O₂ | | | | H₂O₂ | | | | H₂O₂ | | | |
| | concentration | | | | concentration | | | | concentration | | | |
| G | | | | | | | | | | | | |
| H | | | | | | | | | | | | |

### REFERENCES

1. Brenner, S. The genetics of Caenorhabditis elegans. Genetics 77, 71-94 (1974). 75
2. Sulston, J. E. & Brenner, S. The DNA of Caenorhabditis elegans. Genetics 77, 95-104 (1974).
3. Toyoda, Y. et al. Products of the Parkinson's disease-related glyoxalase DJ-1, D-lactate and glycolate, support mitochondrial membrane potential and neuronal survival. Biol. Open 3, 777-784 (2014).
4. Erkut, C., Gade, V. R., Laxman, S. & Kurzchalia, T. V. The glyoxylate shunt is essential for desiccation tolerance in C. Elegans and budding yeast. Elife 5, 1-24 (2016).
5. Flavel, M. R. et al. Growth of Caenorhabditis elegans in Defined Media Is Dependent on Presence of Particulate Matter. G3 8, 567-575 (2018).
6. Caito, S. W. & Aschner, M. Quantification of glutathione in Caenorhabditis elegans. Curr. Protoc. Toxicol. 64, 6.18.1-6.18.6 (2015).
7. Bligh, E. G. and Dyer, W. J. Canadian Journal of Biochemistry and Physiology.

Can. J. Biochem. Physiol. 37, (1959).
8. Ferguson, G. D. & Bridge, W. J. The glutathione system and the related thiol network in Caenorhabditis elegans. Redox Biol. 24, 101171 (2019).
9. Baker, P. R. S., Cramer, S. D., Kennedy, M., Assimos, D. G. & Holmes, R. P. Glycolate and glyoxylate metabolism in HepG2 cells. Am. J. Physiol. Physiol. 287, C1359-C1365 (2004).
10. Holmes, R. P. & Assimos, D. G. Glyoxylate synthesis, and its modulation and influence on oxalate synthesis. J. Urol. 160, 1617-1624 (1998).
11. Salido, E., Pey, A. L., Rodriguez, R. & Lorenzo, V. Primary hyperoxalurias: Disorders of glyoxylate detoxification. Biochim. Biophys. Acta 1822, 1453-1464 (2012).
12. Sawaki, S., Hattori, N. & Yamada, K. Y. Reduction of glyoxylate by lactate dehydrogenase. J. Vitaminol. (Kyoto). 12, 210-213 (1966).
13. Mdluli, K., Booth, M. P. S., Brady, R. L. & Rumsby, G. A preliminary account of the properties of recombinant human Glyoxylate reductase (GRHPR), LDHA and LDHB with glyoxylate, and their potential roles in its metabolism. Biochim. Biophys. Acta—Proteins Proteomics 1753, 209-216 (2005).
14. Sallach, H. J. Formation of serine from hydroxypyruvate and L-alanine*. J. Biol. Chem. 223, 1101-1109 (1956).
15. Kim, W., Underwood, R. S., Greenwald, I. & Shaye, D. D. OrthoList 2: A New Comparative Genomic Analysis of Human and Caenorhabditis elegans Genes. Genetics 210, 445-461 (2018).
16. Greenberg, D. M. & Ichihara, A. Further studies on the pathway of serine formation from carbohydrate. J. Biol. Chem. 224, 331-340 (1957).
17. Do, S.-H., Lee, S.-Y. & Na, H.-S. The effect of repeated isoflurane exposure on serine synthesis pathway during the developmental period in Caenorhabditis elegans. Neurotoxicology 71, 132-137 (2019).
18. Liu, Y. J. et al. Glycine promotes longevity in caenorhabditiselegans in a methionine cycle-dependent fashion. PLoS Genet. 15, 1-23 (2019).
19. Wang, W. et al. Glycine metabolism in animals and humans: implications for nutrition and health. Amino Acids 45, 463-477 (2013).
20. Yilmaz, S. L. & Walhout, A. J. M. A Caenorhabditis elegans Genome-Scale Metabolic Network Model. Cell Syst. 2, 297-311 (2016).
21. Jiang, Z. Y., Lu, M. C. & You, Q. D. Nuclear Factor Erythroid 2-Related Factor 2 (Nrf2) Inhibition: An Emerging Strategy in Cancer Therapy. J. Med. Chem. 62, 3840-3856 (2019).
22. Covas, G., Marinho, H. S., Cyrne, L. & Antunes, F. Activation of Nrf2 by H2O2: De novo synthesis versus nuclear translocation. Methods Enzymol. 528, 157-171 (2013).
23. Naji, A. et al. The activation of the oxidative stress response transcription factor SKN-1 in Caenorhabditis elegans by mitis group streptococci. PLoS One 13, 1-19 (2018).
24. Blackwell, T. K., Steinbaugh, M. J., Hourihan, J. M., Ewald, C. Y. & Isik, M. SKN1/Nrf, stress responses, and aging in Caenorhabditis elegans. Free Radic. Biol.

Med. 88, 290-301 (2015).
25. Ristow, M. & Schmeisser, K. Mitohormesis: Promoting health and lifespan by increased levels of reactive oxygen species (ROS). Dose-Response 12, 288-341 (2014).
26. Bruns, D. R. et al. Nrf2 Signaling and the Slowed Aging Phenotype: Evidence from Long-Lived Models. Oxid. Med. Cell. Longev. 2015, (2015).
27. Townsend, D. M., Tew, K. D. & Tapiero, H. The importance of glutathione in human disease. Biomed Pharmacother. 57, 145-155 (2003).
28. Corpas, F. J. & Barroso, J. B. NADPH-generating dehydrogenases: their role in the mechanism of protection against nitro-oxidative stress induced by adverse environmental conditions. Front. Environ. Sci. 2, 1-5 (2014).
29. Maddocks, O. D. K., Labuschagne, C. F. & Vousden, K. H. Localization of NADPH Production: A Wheel within a Wheel. Mol. Cell 55, 158-160 (2014).
30. Lewis, C. A. et al. Tracing compartmentalized NADPH metabolism in the cytosol and mitochondria of mammalian cells. Mol. Cell 55, 253-263 (2014).
31. Fan, J. et al. Quantitative flux analysisreveals folate-dependent NADPH production. Nature 510, 298-310 (2014).
32. Labuschagne, C. F., van den Broek, N. J. F., Mackay, G. M., Vousden, K. H. & Maddocks, O. D. K. Serine, but not glycine, supports one-carbon metabolism and proliferation of cancer cells. Cell Rep. 7, 1248-1258 (2014).

1. A composition for neutralizing toxic effects of hydrogen peroxide in living cells or tissues, comprising at least two components, a component 1 and a component 2, wherein the component 1 is selected from the group consisting of glycolic acid, glyoxylic acid, glycine, serine or salts thereof and the component 2 is hydrogen peroxide.

2. The composition according to claim 1, wherein the component 1 is selected from the group consisting of glycolic acid, glyoxylic acid, glycine, serine or salts thereof in a concentration range from 0.1 mM to 20 mM, and the component 2 is hydrogen peroxide in a concentration range from 10 ppm to 150 mM.

3. The composition according to claim 1, wherein the glycolic acid, glyoxylic acid, or a salt thereof is presented in a concentration range between 5 mM and 20 mM, and the component 2 is presented in a concentration range from 0.1 mM to 150 mM.

4. The composition according to claim 1, wherein the glycolic acid, glyoxylic acid, or a salt thereof is presented in a concentration range between 9 mM and 11 mM.

5. The composition according to claim 1, wherein the concentration of glycolic acid, glyoxylic acid, or a salt thereof is 10 mM.

6. The composition according to claim 1, wherein glycine is presented in a concentration range between 0.1 mM and 2 mM, and the component 2 is presented in a concentration range from 0.1 mM to 150 mM.

7. The composition according to claim 1, wherein serine is presented in a concentration range between 0.5 mM and 2.0 mM, and the component 2 is presented in a concentration range from 0.1 mM to 150 mM.

8. The composition according to claim 1, further comprising a component 3, the component 3 is selected from the group consisting of rupintrivir, 2-(3,4-dichloro-phenoxy)-5-nitrobenzonitrile, benzothiophenes and ((biphenyloxy)propyl) isoxazoles.

9. The composition according to claim 8, wherein the component 3 is rupintrivir.

10. The composition according to claim 8, wherein the component 3 is contained in the composition in a concentration range from 0.1 mM to 5 mM.

11. The composition according to claim 8, wherein a molar ratio of hydrogen peroxide and the component 3 is in in a range from 1:1 to 1000:1.

12. The composition according to any one of the claims 1 to 11 in a form of a drink solution or a capsule.

13. (canceled)

14. (canceled)

15. (canceled)

16. (canceled)

17. (canceled)

18. (canceled)

19. (canceled)

20. A method of preparing a food product comprising at least two components, a component 1 and a component 2, wherein the component 1 is selected from the group consisting of gycolic acid, glyoxlic acid, glycine, serine or salts thereof and the component 2 is hydrogen peroxide, said method comprising the steps of:
Providing pasteurized milk and mix of bacterial starters for making Joghurt, Matsoni, Dahi, Bathora that comprise Individual bacterial cultures comprising the following bacterial strains: Lactobacillus coryniformis, Lactobacillus delbrueckii, Lactobacillus rhamnosus, Lactococcuslactis, and Streptococcus thermophiles;
mixing the predetermined amount of said starter or bacterial culture with the milk and fermenting the mixture under aerobic conditions by contacting the milk with air;
measuring the hydrogen peroxide concentration in the surface layer after forming the texture of the fermentation product with the thickness of up to substantially 2 cm;
removing the surface layer upon achieving the hydrogen peroxide concentration within the range of from 30 ppm to 50 ppm and refrigerating the surface layer so as to reduce the fermentation process therein;
further fermenting the mass remaining after the step of removing the surface layer by repeating the preceding steps until the last surface layer has been fermented;
combining all layers containing the hydrogen peroxide;
adding the component 1 before or after the step of fermentation.

21. (canceled)

22. (canceled)

23. (canceled)

24. (canceled)

25. (canceled)

26. (canceled)

27. A kit for neutralizing toxic effects of hydrogen peroxide in living cells or tissues, comprising at least two components, a component 1 and a component 2, wherein the component 1 is selected from the group consisting of glycolic acid, glyoxylic acid, glycine, serine or salts thereof, and component 2 is hydrogen peroxide.

28. The kit according to claim 27, wherein the component 1 is selected from the group consisting of glycolic acid, glyoxylic acid, glycine, serine or salts thereof in a concentration range from 0.1 mM to 20 mM, and component 2 is hydrogen peroxide in a concentration range from 10 ppm to 150 mM.

29. The kit according to claim 27, further comprising a component 3, the component 3 being selected from the group consisting of rupintrivir, 2-(3,4-dichloro-phenoxy)-5-nitrobenzonitrile, benzothiophenes and ((biphenyloxy)propyl) isoxazoles.

30. The kit according to claim 29, wherein the component 3 is rupintrivir.

31. The kit according to claim 29, wherein the component 3 is contained in the composition in a concentration range from 0.1 mM to 5 mM.

32. (canceled)

33. The kit for use according to claim 32, for application to a subject in need of prophylaxis or treatment of bacterial and viral diseases through oral, nasal, auricle, topical or rectal rout.

34. The kit for use according to claim 32, wherein the component 1 and component 2 are administered to a subject in need of prophylaxis or treatment of bacterial and viral diseases in sequence, the time period between administering the component 1 and the component 2 being 30 minutes at most.
